SEQUENCE LISTING (17 sequences)

SEQ ID NO: 1 - DNA, 11017 bp, synthetic construct
  source 1..11017: mol_type = other DNA
ctacggctggcccagatccttccctgccgcctccttcaggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtggggccactagggacaggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccatactgcgctaccctctcccagaacctggcttgtcgacgacggcgttctcagtggtgtacggtacaaacccagagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagcttggcttgtcgacgacggcgccctcagtggtgtacggtacaaacccaggtaccgagctcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgacctatataagcagagctcgtttagtgaaccgtcagatcgcctggagacgccatccacgctgttttgacctccatagaagacaccgggaccgatccagcctccgcggccccgaattgcaagtttgtacaaaaaagcaggctgccaccatgttcgtcaaatccgagaccttggagttgaaggaggaagaggacgtgttagtgctgctcggatcggcctcccccgccttggcggccctgaccccgctgtcatccagcgccgacgaagaagaggaggaggagccgggcgcgtcaggcggggcgcgtcggcagcgcggggctgaggccgggcagggggcgcggggcggcgtggctgcgggtgcggagggctgccggcccgcacggctgctgggtctggtacacgattgcaaacggcgcccttcccgggcgcgggccgtctcccgaggcgccaagacggccgagacggtgcagcgcatcaagaagacccgtagactgaaggccaacaaccgcgagcgaaaccgcatgcacaacctcaacgcggcactggacgcgctgcgcgaggtgctccccacgttccccgaggacgccaagctcaccaagatcgagaccctgcgcttcgcccacaactacatctgggcactcaccgagaccctgcgcctggcggatcactgcgggggcggcggcgggggcctgccgggggcgctcttctccgaggcagtgttgctgagcccgggaggcgccagcgccgccctgagcagcagcggagacagcccctcgcccgcctccacgtggagttgcaccaacagccccgcgccgtcctcctccgtgtcctccaattccacctccccctacagctgcactttatcgcccgccagcccggccgggtcagacatggactattggcagcccccacctcccgacaagcaccgctatgcacctcacctccccatagccagggattgtatcttgtgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggacccagctttcttgtacaaagtggctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcaggccaccatggcacacgcaccaggcaccgaccagatgttctacgtgggcacaatggacggctggtatctggataccaagctgaactccgtggccatcggcgcccactggtcttgctttatcgtgctgacaatcaccacattctacctgggctatgagtcctggacatctcggggccctagcaagagaacctccttttacgccggctatcaggaggagcagaacctggccctgtttgtgaatttctttgccatgctgagctacttcggcaagatcgtggccgacacactgggccacaacttcggcgatgtgggccccttcatcatcggcttcggcaattacaggtatgccgactacatgctgacctgcccaatgctggtgtacgatctgctgtatcagctgagggccccctatcgcgtgtcttgtagcgccatcatctttgccatcctgatgtctggcgtgctggccgagttctacgcagagggcgaccctaggctgaggaatggcgcctacgcctggtatggctttggctgtttctggtttatcttcgcctactctatcgtgatgagcatcgtggccaagcagtatagccggctggcccagctggcccaggatacaggcgccgagcacagcctgcacgtgctgaagttcgccgtgtttaccttctccatgctgtggattctgtttcccctggtgtgggccatctgccctagaggcttcggctggatcgacgataactggacagaggtggcccactgcgtgtgcgacatcgtggccaagtcctgttacggctttgccctggcccggttcagaaagacctatgatgaggagctgtttcggctgctggagcagctgggacacgacgaggatgagttccagaagctggagctggatatgaggctgagcagcaatggcgagcgcctgcggagactgtctgcggccgccaagagcaggatcaccagcgagggcgagtacatccccctggaccagatcgacatcaacgtggtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcaccaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaagacaatgggctgggaagcgtccaccgagcggttgtaccccgaggacggcgtgctgaagggcgacattaagatggccctgcgcctgaaggacggcgggcgctacctggcggacttcaagaccacctacaaggccaagaagcccgtgcagatgcccggcgcctacaacgtggaccgcaagttggacatcacctcccacaacgaggactacaccgtggtggaacagtacgaacgctccgagggccgccactccaccggcggcatggacgagctgtacaagcagtcccagcccatcctcaacaccaaggagatggccccgcagagcaagcctccagaggagctggagatgagcagcatgcccagccccgtggcccctctgcccgcacgcacggagggcgtcatcgacatgcggagcatgtccagcattgacagcttcatcagctgtgccacggacttccctgaagccaccagattcttctgctacgagaacgaggtgtaatcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatctgggtttgtaccgtacaccactgaggacgccgtcgtcgacaagccggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacaagtttgtacaaaaaagcaggctgccaccatgtctagattagataaaagtaaagtgattaacagcgcattagagctgcttaatgaggtcggaatcgaaggtttaacaacccgtaaactcgcccagaagctaggtgtagagcagcctacattgtattggcacgtgcgcaacaagcagactcttatgaacatgctttcagaggcaatactggcgaagcatcacacccgttcagcaccgttaccgactgagagttggcagcagtttctccaggaaaatgctctgagtttccgtaaagcattactggtccatcgtgatggagcccgattgcatatagggacctctcctacgcccccccagtttgaacaagcagaggcgcaactacgctgtctatgcgatgcagggttttcggtcgaggaggctcttttcattctgcaatctatcagccattttacgttgggtgcagtattagaggagcaagcaacaaaccagatagaaaataatcatgtgatagacgctgcaccaccattattacaagaggcatttaatattcaggcgagaacctctgctgaaatggccttccatttcgggctgaaatcattaatatttggattttctgcacagttagatgaaaaaaagcatacacccattgaggatggtaataaaccaaaaaagaagagaaagctagcagtgtcagtgacatttgaagatgtggctgtgctctttactcgggacgagtggaagaagctggatctgtctcagagaagcctgtaccgtgaggtgatgctggagaattacagcaacctggcctccatggcaggattcctgtttaccaaaccaaaggtgatctccctgttgcagcaaggagaggatccctggggctccggagagggcaggggaagtcttctaacatgcggggacgtggaggaaaatcccggccccatgtctagactggacaagagcaaagtcataaacggcgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccgggggaagcggagccacgaacttctctctgttaaagcaagcaggagatgttgaagaaaaccccgggcctatgagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacgccaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattgaacccagctttcttgtacaaagtggcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagctttgggtttgtaccgtacaccactgagctcgccgtcgtcgacaagccgagctgctctgacgcggccgtctattgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttctctttctccatccttctttccttaaagagtccccagtgctatctgggacatattcctccgcccagagcagggtcccgcttccctaaggccctgctctgggcttctgggtttgagtccttggcaagcccaggagaggcgctcaggcttccctgtcccccttcctcgtccaccatctcatgcccctggctctcctgccccttccctacaggggttcctggctctgctcttcagactgagccccgttcccctgcatccccgttcccctgcatcccccttcccctgcatcccccagaggccccaggccacctacttggcctggaccccacgagaggccaccccagccctgtctaccaggctgccttttgggtggattctcctccaactgtggggtgactgcttggcaaactcactcttcggggtatcccaggaggcctggagcattggggtgggctggggttcagagaggagggattcccttctcaggttacgtggccaagaagcaggggagctgggtttgggtcaggtctgggtgtggggtgaccagcttatgctgtttgcccaggacagcctagttttagcactgaaaccctcagtcctaggaaaacagggatggttggtcactgtctctgggtgactcttgattcccggccagtttctccacctggggctgtgtttctcgtcctgcatccttctccaggcagg

SEQ ID NO: 2 - DNA, 4793 bp, synthetic construct
  source 1..4793: mol_type = other DNA
catgtatagaggaattatgagggaaagataccaagtcacggtttattcttcaaaatggaggtggcttgttgggaaggtggaagctcatttggccagagtggaaatggaattgggagaaatcgatgaccaaatgtaaacacttggtgcctgatatagcttgacaccaagttagccccaagtgaaataccctggcaatattaatgtgtcttttcccgatattcctcaggtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtggggtaagtcttacattcttttgtaagctgctgaaagttgtgtatgagtagtcatatcataaagctgctttgatataaaaaaggtctatggccatactaccctgaatgagtcccatcccatctgatataaacaatctgcatattgggattgtcagggaatgttcttaaagatcagattagtggcacctgctgagatactgatgcacagcatggtttctgaaccagtagtttccctgcagttgagcagggagcagcagcagcacttgcacaaatacatatacactcttaacacttcttacctactggcttcctctagcttttgtggcagcttcaggtatatttagcactgaacgaacatctcaagaaggtataggcctttgtttgtaagtcctgctgtcctagcatcctataatcctggacttctccagtactttctggctggattggtatctgaggctagtaggaagggcttgttcctgctgggtagctctaaacaatgtattcatgggtaggaacagcagcctattctgccagccttatttctaaccattttagacatttgttagtacatggtattttaaaagtaaaacttaatgtcttccttttttttctccactgtctttttcatagatcgagacatgggaggcggtgggtctggtggcggaggctcgggcggaggtgggtcgggtggcggcggatcaatggtagatggaaccctccttttactcctctcggaggccctggcccttacccagacctgggcgggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgggaagcggagagggcaggggaagtcttctaacatgcggggacgtggaggaaaatcccggccccatgcccacgctactgcgggtttatatagacggtcctcacgggatggggaaaaccaccaccacgcaactgctggtggccctgggttcgcgcgacgatatcgtctacgtacccgagccgatgacttactggcaggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtacaacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccacccccggctccataccgacgatctgcgacctggcgcgcacgtttgcccgggagatgggggaggctaactgagtgcgggtgccagggcgtgcccccgagttctctcagttggggggcagcatcatggaggtaagtttttgaccttgagaaaatgtttttgtttcactgtcctgaggactatttatagacagctctaacatgataaccctcactatgtggagaacattgacagagtaacattttagcagggaaagaagaatcctacagggtcatgttcccttctcctgtggagtggcatgaagaaggtgtatggccccaggtatggccatattactgaccctctacagagagggcaaaggaactgccagtatggtattgcaggataaaggcaggtggttacccacattacctgcaaggctttgatctttcttctgccatttccacattggacatctctgctgaggagagaaaatgaaccactcttttcctttgtataatgttgttttattcttcagacagaagagaggagttatacagctctgcagacatcccattcctgtatggggactgtgtttgcctcttagaggttcccaggccactagaggagataaagggaaacagattgttataacttgatataatgatactataatagatgtaactacaaggagctccagaagcaagagagagggaggaacttggacttctctgcatctttagttggagtccaaaggcttttcaatgaaattctactgcccagggtacattgatgctgaaaccccattcaaatctcctgttatattctagaacagggaattgatttgggagagcatcaggaaggtggatgatctgcccagtcacactgttagtaaattgtagagccaggacctgaactctaatatagtcatgtgttacttaatgacggggacatgttctgagaaatgcttacacaaacctaggtgttgtagcctactacacgcataggctacatggtatagcctattgctcctagactacaaacctgtacagcctgttactgtactgaatactgtgggcagttgtaacacaatggtaagtatttgtgtatctaaacatagaagttgcagtaaaaatatgctattttaatcttatgagaccactgtcatatatacagtccatcattgaccaaaacatcatatcagcattttttcttctaagattttgggagcaccaaagggatacactaacaggatatactctttataatgggtttggagaactgtctgcagctacttcttttaaaaaggtgatctacacagtagaaattagacaagtttggtaatgagatctgcaatccaaataaaataaattcattgctaacctttttcttttcttttcaggtttgaagatgccgcatttggattggatgaattccaaattctgcttgcttgctttttaatattgatatgcttatacacttacactttatgcacaaaatgtagggttataataatgttaacatggacatgatcttctttataattctactt

SEQ ID NO: 3 - DNA, 4632 bp, synthetic construct
  source 1..4632: mol_type = other DNA
ggagttgaagactagcctgagcaacatggcaagatcctgtctcaaaattctttttaaaatttttttaaaagcccaggcttggtggcgcatgcctatagttccagctactcaggaggctgaggcagaaggatctcttgaacccaggagtttcaggctgtagttcactatgatggcagctgtgaatagcctgggcaacacagcaagacctcatctccaaaaaaagacaaaagaactaaattattctactgcagaacatgattaggtaaatatctccaaagcagaaagacaggtttcatattttcgttagtttgagtcagtccttccaaatcaaatcttgttttttattagtatacagatggtatagccagtaagtaaatgagaagcagtctttttaagccgatccattcttaaatgaaaaaatatataaatattttagaataaatttattaaattctaaagttgtagaatttttaaatttggatattttgggaaaatatttaaaccactattgcaaacaaaacaacaaaatgtacttatgtttatacttaggcacaaagaaaactacagtattttaaagtaaccattacacaatattgaggttgcaaagattactgaaggcataacctaaaaaatgagttgatttctaaaaatgggaaaaaggaaaaaaataatttctaaaaacaagtatgcatacctaaacctacctaatgacaccttagaaaattcaagtatagcaccattcattaacatcaatgaggatgtcatcacacatcatgtagcctctgcacaccgtgagaataaatgaaaaagacaggcatcttgctatcatgacaatagttttgacctcgcagacctctctgtgcttacgcaacggataaagccataagaactgtcctgccctcaaggagcaacctaaagtaggaaaaaaaaaacaaaattacacaattattatttacaattgtgagaagagctcttgacaacattcaaatggaggatacagtgtagtaagggtgagggtatcaaggcttccttgagaagtgatgttttgaggccatggcttgtcgacgacggcgacctcagtggtgtacggtacaaacccaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagccaccatgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaaagctgtagaggaaccccttaatgcattcaaagaatcaaaaggaatgatgaatgatgaataacaagtttgtacaaaaaagcaggctcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagtgcgggtgccagggcgtgcccccgagttctctcagttgggggtgggtttgtaccgtacaccactgagcacgccgtcgtcgacaagcctctttcttctcaataactggtatttggttcctgaatcctttaacttccttaccattgtcactcctaagccaaatctcattacgtcatgtctagactactgttaagagaaccacttaagtggtctctgcagccctcaatttattggtgttatctatgggaaaattgctcaaactctaagccttagtttcctaccctataaaatggggttttatatacaaggaacatactaaatacacaggtatacctcagaaacacggcaggctcaattccagagcactacaataaagcgaatctcatgaatttgttggtttcccagtgcataaattatggttacactataccatagtctattaagaagtgtacaatagcattatgtataattgataaatacatcactgctaaaaaaatgctaacaatctttttgctggtggagggtcttacgccaacgttaacgatggctactgactcatcagggtggtggtggttgaagattacgatagctgtggcaatttcttaaaagacaatgaagtttgccacactgacatcctttcacaagacttctctgtagcatgtgatgctgtttgatagcatgatagcattttacccacagtagaacttttttttccttttcctttttttttttttttaaacgcaaggtctcactctgtcacccaggctggagtgcaggggcgccatctcggcttactgcaacctcctcctccctggttcaagagattctcctgcctcagcttcccaagtagctgggactacaggtgtgcaccacacctggctaatttggtagaggtggggtttcaccatgttggcaaggctggtcttgaactcctgacctcaaatgatctaccagtctcggcctcctaaagtactgggattgcaggtgtgagccaccacacccagccagtggaacttatttcaaaattgaagtcaactctctcacaccctggcactgctttatcaaccaggtttctgtaattcctaaatcctttattggcattttaacaatgttcacagcaacttcaccag

SEQ ID NO: 4 - DNA, 13950 bp, synthetic construct
  source 1..13950: mol_type = other DNA
ctacggctggcccagatccttccctgccgcctccttcaggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtggggccactagggacaggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccatactgcgctaccctctcccagaacctggcttgtcgacgacggcgttctcagtggtgtacggtacaaacccagagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagcttggcttgtcgacgacggcgccctcagtggtgtacggtacaaacccaggtaccgagctcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgacctatataagcagagctcgtttagtgaaccgtcagatcgcctggagacgccatccacgctgttttgacctccatagaagacaccgggaccgatccagcctccgcggccccgaattgcaagtttgtacaaaaaagcaggctgccaccatgttcgtcaaatccgagaccttggagttgaaggaggaagaggacgtgttagtgctgctcggatcggcctcccccgccttggcggccctgaccccgctgtcatccagcgccgacgaagaagaggaggaggagccgggcgcgtcaggcggggcgcgtcggcagcgcggggctgaggccgggcagggggcgcggggcggcgtggctgcgggtgcggagggctgccggcccgcacggctgctgggtctggtacacgattgcaaacggcgcccttcccgggcgcgggccgtctcccgaggcgccaagacggccgagacggtgcagcgcatcaagaagacccgtagactgaaggccaacaaccgcgagcgaaaccgcatgcacaacctcaacgcggcactggacgcgctgcgcgaggtgctccccacgttccccgaggacgccaagctcaccaagatcgagaccctgcgcttcgcccacaactacatctgggcactcaccgagaccctgcgcctggcggatcactgcgggggcggcggcgggggcctgccgggggcgctcttctccgaggcagtgttgctgagcccgggaggcgccagcgccgccctgagcagcagcggagacagcccctcgcccgcctccacgtggagttgcaccaacagccccgcgccgtcctcctccgtgtcctccaattccacctccccctacagctgcactttatcgcccgccagcccggccgggtcagacatggactattggcagcccccacctcccgacaagcaccgctatgcacctcacctccccatagccagggattgtatcttgtgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcaggccaccatggcacacgcaccaggcaccgaccagatgttctacgtgggcacaatggacggctggtatctggataccaagctgaactccgtggccatcggcgcccactggtcttgctttatcgtgctgacaatcaccacattctacctgggctatgagtcctggacatctcggggccctagcaagagaacctccttttacgccggctatcaggaggagcagaacctggccctgtttgtgaatttctttgccatgctgagctacttcggcaagatcgtggccgacacactgggccacaacttcggcgatgtgggccccttcatcatcggcttcggcaattacaggtatgccgactacatgctgacctgcccaatgctggtgtacgatctgctgtatcagctgagggccccctatcgcgtgtcttgtagcgccatcatctttgccatcctgatgtctggcgtgctggccgagttctacgcagagggcgaccctaggctgaggaatggcgcctacgcctggtatggctttggctgtttctggtttatcttcgcctactctatcgtgatgagcatcgtggccaagcagtatagccggctggcccagctggcccaggatacaggcgccgagcacagcctgcacgtgctgaagttcgccgtgtttaccttctccatgctgtggattctgtttcccctggtgtgggccatctgccctagaggcttcggctggatcgacgataactggacagaggtggcccactgcgtgtgcgacatcgtggccaagtcctgttacggctttgccctggcccggttcagaaagacctatgatgaggagctgtttcggctgctggagcagctgggacacgacgaggatgagttccagaagctggagctggatatgaggctgagcagcaatggcgagcgcctgcggagactgtctgcggccgccaagagcaggatcaccagcgagggcgagtacatccccctggaccagatcgacatcaacgtggtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcaccaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaagacaatgggctgggaagcgtccaccgagcggttgtaccccgaggacggcgtgctgaagggcgacattaagatggccctgcgcctgaaggacggcgggcgctacctggcggacttcaagaccacctacaaggccaagaagcccgtgcagatgcccggcgcctacaacgtggaccgcaagttggacatcacctcccacaacgaggactacaccgtggtggaacagtacgaacgctccgagggccgccactccaccggcggcatggacgagctgtacaagcagtcccagcccatcctcaacaccaaggagatggccccgcagagcaagcctccagaggagctggagatgagcagcatgcccagccccgtggcccctctgcccgcacgcacggagggcgtcatcgacatgcggagcatgtccagcattgacagcttcatcagctgtgccacggacttccctgaagccaccagattcttctgctacgagaacgaggtgtaatcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcacccagctttcttgtacaaagtggtaccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgttattcatcattcatcattccttttgattctttgaatgcattaaggggttcctctacagctttcctaggaggttgtatagtcttctgattggaagccacaaatttcatataaactagtccaagtaattgtgctagagctaagatactcaaacctgaaatcagaagagggccatgcattggtatacacgccgcaatacagagactcagtccaaccacagcgaggatataggctatcacctgaataaccaatatggcaatgacgaaggaggttaatccaatcgctgtactaaacacatagtagtgaagtaatattaatatccctgtagaagtcacaattaaaccaaggccagtagcattctttaatgaatattcacctgggacgaaaagaatggctccaacaatgacaatgacagtgatcactagtccagcaacaagtaaagcaattgttttctcatccataccaccggatctatatttaagtgttttaataccaaactgtccccagaacaggagtatagcaaaaattgggaaaataacaataagaatattttcatttggagaaaaccatgaaacaacacgatattttagctcgatgatcgtttcaccttctctggttaattctgttacttcacaagtgtagtttcctgtgtgtgagacagcatcactcttatccatcttcaaagaggcatctccttttagtaattgtgagacttcaatttttgcactactaaagtcagtggggacagtggacttgtttagagctccatcaaaggtgtaaatatctcttcctttaaatttccactttacgtatacttcagtagtgttttgtgcctccatattagtaacaaagcatggaatgacgacagtgtcattacaaaacgtgaattctacagattttgttttattaaatagtagctgagctgatccgcagcacgccgagcccagcaacagcgccgctaccaggggccacatggtggccaattctttgccaaaatgatgagacagcacaataaccagcacgttgcccaggagctgtaggaaaaagaagaaggcatgaacatggttagcagaggctctagagccgccggtcacacgccagaagccgaaccccgccctgccccgtcccccccgaaggcagccgtccccccgcggacagccccgaggctggagagggagaaggggacggcggcgcggcgacgcacgaaggccctccccgcccatttccttcctgccggcgccgcaccgcttcgccccgcgcccgctagagggggtgcggcggcgcctcccagatttcggctccgcacagatttgggacaaaggaagtccctgcgccctctcgcacgattaccataaaaggcaatggctgcggctcgccgcgcctcgacagccgccggcgctccgggggccgccgcgcccctcccccgagccctccccggcccgaggcggccccgccccgcccggcacccccacctgccgccaccccccgcccggcacggcgagccccgcgccacgccccgtacggagccccgcacccgaagccgggccgtgctcagcaactcggggaggggggtgcagggggggttgcagcccgaccgacgcgcccacaccccctgctcacccccccacgcacacaccccgcacgcagcctttgttcccctcgcagccccccccgcaccgcggggcaccgcccccggccgcgctcccctcgcgcacactgcggagcgcacaaagccccgcgccgcgcccgcagcgctcacagccgccgggcagcgcggagccgcacgcggcgctccccacgcacacacacacgcacgcaccccccgagccgctccccccgcacaaagggccctcccggagcccctcaaggctttcacgcagccacagaaaagaaacaagccgtcattaaaccaagcgctaattacagcccggaggagaagggccgtcccgcccgctcacctgtgggagtaacgcggtcagtcagagccggggcgggcggcgcgaggcggcggcggagcggggcacggggcgaaggcagcgcgcagcgactcccgcccgccgcgcgcttcgctttttatagggccgccgccgccgccgcctcgccataaaaggaaactttcggagcgcgccgctctgattggctgccgccgcacctctccgcctcgccccgccccgcccctcgccccgccccgccccgcctggcgcgcgccccccccccccccccgcccccatcgctgcacaaaataattaaaaaataaataaatacaaaattgggggtggggaggggggggagatggggagagtgaagcagaacgtggggctcacctcgaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcgagtgggtttgtaccgtacaccactgaggacgccgtcgtcgacaagccggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacaagtttgtacaaaaaagcaggctgccaccatgtctagattagataaaagtaaagtgattaacagcgcattagagctgcttaatgaggtcggaatcgaaggtttaacaacccgtaaactcgcccagaagctaggtgtagagcagcctacattgtattggcacgtgcgcaacaagcagactcttatgaacatgctttcagaggcaatactggcgaagcatcacacccgttcagcaccgttaccgactgagagttggcagcagtttctccaggaaaatgctctgagtttccgtaaagcattactggtccatcgtgatggagcccgattgcatatagggacctctcctacgcccccccagtttgaacaagcagaggcgcaactacgctgtctatgcgatgcagggttttcggtcgaggaggctcttttcattctgcaatctatcagccattttacgttgggtgcagtattagaggagcaagcaacaaaccagatagaaaataatcatgtgatagacgctgcaccaccattattacaagaggcatttaatattcaggcgagaacctctgctgaaatggccttccatttcgggctgaaatcattaatatttggattttctgcacagttagatgaaaaaaagcatacacccattgaggatggtaataaaccaaaaaagaagagaaagctagcagtgtcagtgacatttgaagatgtggctgtgctctttactcgggacgagtggaagaagctggatctgtctcagagaagcctgtaccgtgaggtgatgctggagaattacagcaacctggcctccatggcaggattcctgtttaccaaaccaaaggtgatctccctgttgcagcaaggagaggatccctggggctccggagagggcaggggaagtcttctaacatgcggggacgtggaggaaaatcccggccccatgtctagactggacaagagcaaagtcataaacggcgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccgggggaagcggagccacgaacttctctctgttaaagcaagcaggagatgttgaagaaaaccccgggcctatgagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacgccaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattgaacccagctttcttgtacaaagtggcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagctttgggtttgtaccgtacaccactgagctcgccgtcgtcgacaagccgagctgctctgacgcggccgtctattgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttctctttctccatccttctttccttaaagagtccccagtgctatctgggacatattcctccgcccagagcagggtcccgcttccctaaggccctgctctgggcttctgggtttgagtccttggcaagcccaggagaggcgctcaggcttccctgtcccccttcctcgtccaccatctcatgcccctggctctcctgccccttccctacaggggttcctggctctgctcttcagactgagccccgttcccctgcatccccgttcccctgcatcccccttcccctgcatcccccagaggccccaggccacctacttggcctggaccccacgagaggccaccccagccctgtctaccaggctgccttttgggtggattctcctccaactgtggggtgactgcttggcaaactcactcttcggggtatcccaggaggcctggagcattggggtgggctggggttcagagaggagggattcccttctcaggttacgtggccaagaagcaggggagctgggtttgggtcaggtctgggtgtggggtgaccagcttatgctgtttgcccaggacagcctagttttagcactgaaaccctcagtcctaggaaaacagggatggttggtcactgtctctgggtgactcttgattcccggccagtttctccacctggggctgtgtttctcgtcctgcatccttctccaggcagg

SEQ ID NO: 5 - DNA, 8974 bp, synthetic construct
  source 1..8974: mol_type = other DNA
ggcttgtcgacgacggcgttctcagtggtgtacggtacaaacccagagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagcttggcttgtcgacgacggcgccctcagtggtgtacggtacaaacccaggtaccgagctcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgacctatataagcagagctcgtttagtgaaccgtcagatcgcctggagacgccatccacgctgttttgacctccatagaagacaccgggaccgatccagcctccgcggccccgaattgcaagtttgtacaaaaaagcaggctgccaccatgttcgtcaaatccgagaccttggagttgaaggaggaagaggacgtgttagtgctgctcggatcggcctcccccgccttggcggccctgaccccgctgtcatccagcgccgacgaagaagaggaggaggagccgggcgcgtcaggcggggcgcgtcggcagcgcggggctgaggccgggcagggggcgcggggcggcgtggctgcgggtgcggagggctgccggcccgcacggctgctgggtctggtacacgattgcaaacggcgcccttcccgggcgcgggccgtctcccgaggcgccaagacggccgagacggtgcagcgcatcaagaagacccgtagactgaaggccaacaaccgcgagcgaaaccgcatgcacaacctcaacgcggcactggacgcgctgcgcgaggtgctccccacgttccccgaggacgccaagctcaccaagatcgagaccctgcgcttcgcccacaactacatctgggcactcaccgagaccctgcgcctggcggatcactgcgggggcggcggcgggggcctgccgggggcgctcttctccgaggcagtgttgctgagcccgggaggcgccagcgccgccctgagcagcagcggagacagcccctcgcccgcctccacgtggagttgcaccaacagccccgcgccgtcctcctccgtgtcctccaattccacctccccctacagctgcactttatcgcccgccagcccggccgggtcagacatggactattggcagcccccacctcccgacaagcaccgctatgcacctcacctccccatagccagggattgtatcttgtgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggacccagctttcttgtacaaagtggctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcaggccaccatggcacacgcaccaggcaccgaccagatgttctacgtgggcacaatggacggctggtatctggataccaagctgaactccgtggccatcggcgcccactggtcttgctttatcgtgctgacaatcaccacattctacctgggctatgagtcctggacatctcggggccctagcaagagaacctccttttacgccggctatcaggaggagcagaacctggccctgtttgtgaatttctttgccatgctgagctacttcggcaagatcgtggccgacacactgggccacaacttcggcgatgtgggccccttcatcatcggcttcggcaattacaggtatgccgactacatgctgacctgcccaatgctggtgtacgatctgctgtatcagctgagggccccctatcgcgtgtcttgtagcgccatcatctttgccatcctgatgtctggcgtgctggccgagttctacgcagagggcgaccctaggctgaggaatggcgcctacgcctggtatggctttggctgtttctggtttatcttcgcctactctatcgtgatgagcatcgtggccaagcagtatagccggctggcccagctggcccaggatacaggcgccgagcacagcctgcacgtgctgaagttcgccgtgtttaccttctccatgctgtggattctgtttcccctggtgtgggccatctgccctagaggcttcggctggatcgacgataactggacagaggtggcccactgcgtgtgcgacatcgtggccaagtcctgttacggctttgccctggcccggttcagaaagacctatgatgaggagctgtttcggctgctggagcagctgggacacgacgaggatgagttccagaagctggagctggatatgaggctgagcagcaatggcgagcgcctgcggagactgtctgcggccgccaagagcaggatcaccagcgagggcgagtacatccccctggaccagatcgacatcaacgtggtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcaccaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaagacaatgggctgggaagcgtccaccgagcggttgtaccccgaggacggcgtgctgaagggcgacattaagatggccctgcgcctgaaggacggcgggcgctacctggcggacttcaagaccacctacaaggccaagaagcccgtgcagatgcccggcgcctacaacgtggaccgcaagttggacatcacctcccacaacgaggactacaccgtggtggaacagtacgaacgctccgagggccgccactccaccggcggcatggacgagctgtacaagcagtcccagcccatcctcaacaccaaggagatggccccgcagagcaagcctccagaggagctggagatgagcagcatgcccagccccgtggcccctctgcccgcacgcacggagggcgtcatcgacatgcggagcatgtccagcattgacagcttcatcagctgtgccacggacttccctgaagccaccagattcttctgctacgagaacgaggtgtaatcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatctgggtttgtaccgtacaccactgaggacgccgtcgtcgacaagccggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacaagtttgtacaaaaaagcaggctgccaccatgtctagattagataaaagtaaagtgattaacagcgcattagagctgcttaatgaggtcggaatcgaaggtttaacaacccgtaaactcgcccagaagctaggtgtagagcagcctacattgtattggcacgtgcgcaacaagcagactcttatgaacatgctttcagaggcaatactggcgaagcatcacacccgttcagcaccgttaccgactgagagttggcagcagtttctccaggaaaatgctctgagtttccgtaaagcattactggtccatcgtgatggagcccgattgcatatagggacctctcctacgcccccccagtttgaacaagcagaggcgcaactacgctgtctatgcgatgcagggttttcggtcgaggaggctcttttcattctgcaatctatcagccattttacgttgggtgcagtattagaggagcaagcaacaaaccagatagaaaataatcatgtgatagacgctgcaccaccattattacaagaggcatttaatattcaggcgagaacctctgctgaaatggccttccatttcgggctgaaatcattaatatttggattttctgcacagttagatgaaaaaaagcatacacccattgaggatggtaataaaccaaaaaagaagagaaagctagcagtgtcagtgacatttgaagatgtggctgtgctctttactcgggacgagtggaagaagctggatctgtctcagagaagcctgtaccgtgaggtgatgctggagaattacagcaacctggcctccatggcaggattcctgtttaccaaaccaaaggtgatctccctgttgcagcaaggagaggatccctggggctccggagagggcaggggaagtcttctaacatgcggggacgtggaggaaaatcccggccccatgtctagactggacaagagcaaagtcataaacggcgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccgggggaagcggagccacgaacttctctctgttaaagcaagcaggagatgttgaagaaaaccccgggcctatgagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacgccaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattgaacccagctttcttgtacaaagtggcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagctttgggtttgtaccgtacaccactgagctcgccgtcgtcgacaagcc

SEQ ID NO: 6 - DNA, 2236 bp, synthetic construct
  source 1..2236: mol_type = other DNA
ggaggcggtgggtctggtggcggaggctcgggcggaggtgggtcgggtggcggcggatcaatggtagatggaaccctccttttactcctctcggaggccctggcccttacccagacctgggcgggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgggaagcggagagggcaggggaagtcttctaacatgcggggacgtggaggaaaatcccggccccatgcccacgctactgcgggtttatatagacggtcctcacgggatggggaaaaccaccaccacgcaactgctggtggccctgggttcgcgcgacgatatcgtctacgtacccgagccgatgacttactggcaggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtacaacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccacccccggctccataccgacgatctgcgacctggcgcgcacgtttgcccgggagatgggggaggctaactgagtgcgggtgccagggcgtgcccccgagttctctcagttggggg

SEQ ID NO: 7 - DNA, 2536 bp, synthetic construct
  source 1..2536: mol_type = other DNA
ggcttgtcgacgacggcgacctcagtggtgtacggtacaaacccaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagccaccatgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaaagctgtagaggaaccccttaatgcattcaaagaatcaaaaggaatgatgaatgatgaataacaagtttgtacaaaaaagcaggctcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagtgcgggtgccagggcgtgcccccgagttctctcagttgggggtgggtttgtaccgtacaccactgagcacgccgtcgtcgacaagcc

SEQ ID NO: 8 - DNA, 11907 bp, synthetic construct
  source 1..11907: mol_type = other DNA
ggcttgtcgacgacggcgttctcagtggtgtacggtacaaacccagagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagcttggcttgtcgacgacggcgccctcagtggtgtacggtacaaacccaggtaccgagctcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgacctatataagcagagctcgtttagtgaaccgtcagatcgcctggagacgccatccacgctgttttgacctccatagaagacaccgggaccgatccagcctccgcggccccgaattgcaagtttgtacaaaaaagcaggctgccaccatgttcgtcaaatccgagaccttggagttgaaggaggaagaggacgtgttagtgctgctcggatcggcctcccccgccttggcggccctgaccccgctgtcatccagcgccgacgaagaagaggaggaggagccgggcgcgtcaggcggggcgcgtcggcagcgcggggctgaggccgggcagggggcgcggggcggcgtggctgcgggtgcggagggctgccggcccgcacggctgctgggtctggtacacgattgcaaacggcgcccttcccgggcgcgggccgtctcccgaggcgccaagacggccgagacggtgcagcgcatcaagaagacccgtagactgaaggccaacaaccgcgagcgaaaccgcatgcacaacctcaacgcggcactggacgcgctgcgcgaggtgctccccacgttccccgaggacgccaagctcaccaagatcgagaccctgcgcttcgcccacaactacatctgggcactcaccgagaccctgcgcctggcggatcactgcgggggcggcggcgggggcctgccgggggcgctcttctccgaggcagtgttgctgagcccgggaggcgccagcgccgccctgagcagcagcggagacagcccctcgcccgcctccacgtggagttgcaccaacagccccgcgccgtcctcctccgtgtcctccaattccacctccccctacagctgcactttatcgcccgccagcccggccgggtcagacatggactattggcagcccccacctcccgacaagcaccgctatgcacctcacctccccatagccagggattgtatcttgtgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcaggccaccatggcacacgcaccaggcaccgaccagatgttctacgtgggcacaatggacggctggtatctggataccaagctgaactccgtggccatcggcgcccactggtcttgctttatcgtgctgacaatcaccacattctacctgggctatgagtcctggacatctcggggccctagcaagagaacctccttttacgccggctatcaggaggagcagaacctggccctgtttgtgaatttctttgccatgctgagctacttcggcaagatcgtggccgacacactgggccacaacttcggcgatgtgggccccttcatcatcggcttcggcaattacaggtatgccgactacatgctgacctgcccaatgctggtgtacgatctgctgtatcagctgagggccccctatcgcgtgtcttgtagcgccatcatctttgccatcctgatgtctggcgtgctggccgagttctacgcagagggcgaccctaggctgaggaatggcgcctacgcctggtatggctttggctgtttctggtttatcttcgcctactctatcgtgatgagcatcgtggccaagcagtatagccggctggcccagctggcccaggatacaggcgccgagcacagcctgcacgtgctgaagttcgccgtgtttaccttctccatgctgtggattctgtttcccctggtgtgggccatctgccctagaggcttcggctggatcgacgataactggacagaggtggcccactgcgtgtgcgacatcgtggccaagtcctgttacggctttgccctggcccggttcagaaagacctatgatgaggagctgtttcggctgctggagcagctgggacacgacgaggatgagttccagaagctggagctggatatgaggctgagcagcaatggcgagcgcctgcggagactgtctgcggccgccaagagcaggatcaccagcgagggcgagtacatccccctggaccagatcgacatcaacgtggtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcaccaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaagacaatgggctgggaagcgtccaccgagcggttgtaccccgaggacggcgtgctgaagggcgacattaagatggccctgcgcctgaaggacggcgggcgctacctggcggacttcaagaccacctacaaggccaagaagcccgtgcagatgcccggcgcctacaacgtggaccgcaagttggacatcacctcccacaacgaggactacaccgtggtggaacagtacgaacgctccgagggccgccactccaccggcggcatggacgagctgtacaagcagtcccagcccatcctcaacaccaaggagatggccccgcagagcaagcctccagaggagctggagatgagcagcatgcccagccccgtggcccctctgcccgcacgcacggagggcgtcatcgacatgcggagcatgtccagcattgacagcttcatcagctgtgccacggacttccctgaagccaccagattcttctgctacgagaacgaggtgtaatcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcacccagctttcttgtacaaagtggtaccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgttattcatcattcatcattccttttgattctttgaatgcattaaggggttcctctacagctttcctaggaggttgtatagtcttctgattggaagccacaaatttcatataaactagtccaagtaattgtgctagagctaagatactcaaacctgaaatcagaagagggccatgcattggtatacacgccgcaatacagagactcagtccaaccacagcgaggatataggctatcacctgaataaccaatatggcaatgacgaaggaggttaatccaatcgctgtactaaacacatagtagtgaagtaatattaatatccctgtagaagtcacaattaaaccaaggccagtagcattctttaatgaatattcacctgggacgaaaagaatggctccaacaatgacaatgacagtgatcactagtccagcaacaagtaaagcaattgttttctcatccataccaccggatctatatttaagtgttttaataccaaactgtccccagaacaggagtatagcaaaaattgggaaaataacaataagaatattttcatttggagaaaaccatgaaacaacacgatattttagctcgatgatcgtttcaccttctctggttaattctgttacttcacaagtgtagtttcctgtgtgtgagacagcatcactcttatccatcttcaaagaggcatctccttttagtaattgtgagacttcaatttttgcactactaaagtcagtggggacagtggacttgtttagagctccatcaaaggtgtaaatatctcttcctttaaatttccactttacgtatacttcagtagtgttttgtgcctccatattagtaacaaagcatggaatgacgacagtgtcattacaaaacgtgaattctacagattttgttttattaaatagtagctgagctgatccgcagcacgccgagcccagcaacagcgccgctaccaggggccacatggtggccaattctttgccaaaatgatgagacagcacaataaccagcacgttgcccaggagctgtaggaaaaagaagaaggcatgaacatggttagcagaggctctagagccgccggtcacacgccagaagccgaaccccgccctgccccgtcccccccgaaggcagccgtccccccgcggacagccccgaggctggagagggagaaggggacggcggcgcggcgacgcacgaaggccctccccgcccatttccttcctgccggcgccgcaccgcttcgccccgcgcccgctagagggggtgcggcggcgcctcccagatttcggctccgcacagatttgggacaaaggaagtccctgcgccctctcgcacgattaccataaaaggcaatggctgcggctcgccgcgcctcgacagccgccggcgctccgggggccgccgcgcccctcccccgagccctccccggcccgaggcggccccgccccgcccggcacccccacctgccgccaccccccgcccggcacggcgagccccgcgccacgccccgtacggagccccgcacccgaagccgggccgtgctcagcaactcggggaggggggtgcagggggggttgcagcccgaccgacgcgcccacaccccctgctcacccccccacgcacacaccccgcacgcagcctttgttcccctcgcagccccccccgcaccgcggggcaccgcccccggccgcgctcccctcgcgcacactgcggagcgcacaaagccccgcgccgcgcccgcagcgctcacagccgccgggcagcgcggagccgcacgcggcgctccccacgcacacacacacgcacgcaccccccgagccgctccccccgcacaaagggccctcccggagcccctcaaggctttcacgcagccacagaaaagaaacaagccgtcattaaaccaagcgctaattacagcccggaggagaagggccgtcccgcccgctcacctgtgggagtaacgcggtcagtcagagccggggcgggcggcgcgaggcggcggcggagcggggcacggggcgaaggcagcgcgcagcgactcccgcccgccgcgcgcttcgctttttatagggccgccgccgccgccgcctcgccataaaaggaaactttcggagcgcgccgctctgattggctgccgccgcacctctccgcctcgccccgccccgcccctcgccccgccccgccccgcctggcgcgcgccccccccccccccccgcccccatcgctgcacaaaataattaaaaaataaataaatacaaaattgggggtggggaggggggggagatggggagagtgaagcagaacgtggggctcacctcgaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcgagtgggtttgtaccgtacaccactgaggacgccgtcgtcgacaagccggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacaagtttgtacaaaaaagcaggctgccaccatgtctagattagataaaagtaaagtgattaacagcgcattagagctgcttaatgaggtcggaatcgaaggtttaacaacccgtaaactcgcccagaagctaggtgtagagcagcctacattgtattggcacgtgcgcaacaagcagactcttatgaacatgctttcagaggcaatactggcgaagcatcacacccgttcagcaccgttaccgactgagagttggcagcagtttctccaggaaaatgctctgagtttccgtaaagcattactggtccatcgtgatggagcccgattgcatatagggacctctcctacgcccccccagtttgaacaagcagaggcgcaactacgctgtctatgcgatgcagggttttcggtcgaggaggctcttttcattctgcaatctatcagccattttacgttgggtgcagtattagaggagcaagcaacaaaccagatagaaaataatcatgtgatagacgctgcaccaccattattacaagaggcatttaatattcaggcgagaacctctgctgaaatggccttccatttcgggctgaaatcattaatatttggattttctgcacagttagatgaaaaaaagcatacacccattgaggatggtaataaaccaaaaaagaagagaaagctagcagtgtcagtgacatttgaagatgtggctgtgctctttactcgggacgagtggaagaagctggatctgtctcagagaagcctgtaccgtgaggtgatgctggagaattacagcaacctggcctccatggcaggattcctgtttaccaaaccaaaggtgatctccctgttgcagcaaggagaggatccctggggctccggagagggcaggggaagtcttctaacatgcggggacgtggaggaaaatcccggccccatgtctagactggacaagagcaaagtcataaacggcgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccgggggaagcggagccacgaacttctctctgttaaagcaagcaggagatgttgaagaaaaccccgggcctatgagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacgccaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattgaacccagctttcttgtacaaagtggcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagctttgggtttgtaccgtacaccactgagctcgccgtcgtcgacaagcc

SEQ ID NO: 9 - DNA, 2468 bp, Homo sapiens
  source 1..2468: mol_type = other DNA
ttgtgtccggcttcttcacttagcattttttttaaggttcatccatgttccagcatgtatcagtgctttctccctttaatggtggaatactttattgtatagataagcaacattttgtttatccatttattttttggtggatatttgggctgtttttatattttggctattttgaattatccttctgtgaacattcgtgtataggtttttgtgtggatatatctggtttttcttgttttcttttgttttattaatacataatatgatacatatttatggggtatatgtgaatatttattacatgcatagaaggtataatgatcatgtcaggatatttgaggtatccacatttgggattgtttaaagattaaatgaaatagtgttaaaagtatttaatatgcccttcaacaaatgatgaggaaatcttagaatctgctcagactccttcagtttacatattaggaaactgaggcacagaaaggagcagagacttgctcaagtccacccaaagcagtagagcattgtggttaaatgcaggacttcagtcagactgtctgggttcaaatcctggttccacttggacatgggtttccttacataaatcacttcacctctctgagcctcagttttctcatatgcaaagtgaggataataataataccttccttacatggttactgatatgagtattaaatgtgccagctcatgtgcctggcgtataggaggtgctttataaaccttagctgttaccactcatggcattgccaaatgtgggacgggtctcctgactctctggtgtgagattgatggaatccacactttccagttcccttttctacctcctgggtatcttctcatatggttgtaagttccttggaggaagggaatgtggcttgctctctccaccacgctgagcatataagaggtgctgaatgagcgcttttattcactcctctcatccccagccctcaccagctgggagttgttgtaggtgtcaattttctgcctctttccaacaccctgtgaggtgactgagcattgtcttccctcccaggcagctcacagtgtgccaccatggagttggggcccctagaaggtggctacctggagcttcttaacagcgatgctgaccccctgtgcctctaccacttctatgaccagatggacctggctggagaagaagagattgagctctactcaggtgggccctcctccctctggtctcttccggtatcccccacccctcagcttgctgtagagacggcaatcaggggaaattctggtccctgccctcccgtcagcaccacggacagctcccacgtctgtgggacgctctctgcagatggggatgatctcccagccctgccccgcctctccctcgttccccaccagccctctttccagaaatttccttcttcatccaagggacttttcctcccagaacccgacacagacaccatcaactgcgaccagttcagcaggctgttgtgtgacatggaaggtgatgaagagaccagggaggcttatgccaatatcggtgaggaagcacctgagcccagaaaaggacaatcaagggcaagagttctttgctgccacttgtcaatatcacccattcatcatgagccacgtcagtcccctcccacagaaatcattgcaagggggatgcggagcaatggctggaggaacggagactccagggaagagaggggagatggaggccagtgggggaaataggccccttcactaatgaccaccaagaaaacaaaatctcatgtttacatcctccacctccatttctatacgcatttctgcttcttgctcttctgtccatcctttctacaaagcccataccatacacccctttcccttttcctcccagctccttagccaagctactctagtatttgtaataactagcatttactggatactcatagtatgctcattgctgtccatacattatcatctggaatccttacaacaacaaaggaaatacatattattagtttctgtcttttacaaatgaggaaattgaggtttagagaggtgaaacgacctacccaagtttgcattagagatagtaatgtacagatctgaaattctaacctaggtctttctcgtttcaaagtctccatttttaaccacaaaaaaagaaagtgtctggggttggaagatttgctaaggcttaagcagagaagctaatttgtagccttaagaagagctacagctttcagacaagggcaggactcttggggagaggtggtggtggtgttgctgctagtggtttgttttttgggggagagacgtgggatagagaatctcttctcaccagcatcaccaccacctcatacagagcctatgttgaaatagaaaaaagtggcctttttctggaggatgcagcccctaccaggaagtaactgcttggctagcaaagtgcaagccggagtcctgcccccattttccttgtt

SEQ ID NO: 10 - DNA, 2430 bp, synthetic construct
  source 1..2430: mol_type = other DNA
ttgtgtccggcttcttcacttagcattttttttaaggttcatccatgttccagcatgtatcagtgctttctccctttaatggtggaatactttattgtatagataagcaacattttgtttatccatttattttttggtggatatttgggctgtttttatattttggctattttgaattatccttctgtgaacattcgtgtataggtttttgtgtggatatatctggtttttcttgttttcttttgttttattaatacataatatgatacatatttatggggtatatgtgaatatttattacatgcatagaaggtataatgatcatgtcaggatatttgaggtatccacatttgggattgtttaaagattaaatgaaatagtgttaaaagtatttaatatgcccttcaacaaatgatgaggaaatcttagaatctgctcagactccttcagtttacatattaggaaactgaggcacagaaaggagcagagacttgctcaagtccacccaaagcagtagagcattgtggttaaatgcaggacttcagtcagactgtctgggttcaaatcctggttccacttggacatgggtttccttacataaatcacttcacctctctgagcctcagttttctcatatgcaaagtgaggataataataataccttccttacatggttactgatatgagtattaaatgtgccagctcatgtgcctggcgtataggaggtgctttataaaccttagctgttaccactcatggcattgccaaatgtgggacgggtctcctgactctctggtgtgagattgatggaatccacactttccagttcccttttctacctcctgggtatcttctcatatggttgtaagttccttggaggaagggaatgtggcttgctctctccaccacgctgagcatataagaggtgctgaatgagcgcttttattcactcctctcatccccagccctcaccagctgggagttgttgtaggtgtcaattttctgcctctttccaacaccctgtgaggtgactgagcattgtcttccctcccaggcagctcacagtgtgccaccatggagttggggcccctaccccctgtgcctctaccacttctatgaccagatggacctggctggagaagaagagattgagctctactcaggtgggccctcctccctctggtctcttccggtatcccccacccctcagcttgctgtagagacggcaatcaggggaaattctggtccctgccctcccgtcagcaccacggacagctcccacgtctgtgggacgctctctgcagatggggatgatctcccagccctgccccgcctctccctcgttccccaccagccctctttccagaaatttccttcttcatccaagggacttttcctcccagaacccgacacagacaccatcaactgcgaccagttcagcaggctgttgtgtgacatggaaggtgatgaagagaccagggaggcttatgccaatatcggtgaggaagcacctgagcccagaaaaggacaatcaagggcaagagttctttgctgccacttgtcaatatcacccattcatcatgagccacgtcagtcccctcccacagaaatcattgcaagggggatgcggagcaatggctggaggaacggagactccagggaagagaggggagatggaggccagtgggggaaataggccccttcactaatgaccaccaagaaaacaaaatctcatgtttacatcctccacctccatttctatacgcatttctgcttcttgctcttctgtccatcctttctacaaagcccataccatacacccctttcccttttcctcccagctccttagccaagctactctagtatttgtaataactagcatttactggatactcatagtatgctcattgctgtccatacattatcatctggaatccttacaacaacaaaggaaatacatattattagtttctgtcttttacaaatgaggaaattgaggtttagagaggtgaaacgacctacccaagtttgcattagagatagtaatgtacagatctgaaattctaacctaggtctttctcgtttcaaagtctccatttttaaccacaaaaaaagaaagtgtctggggttggaagatttgctaaggcttaagcagagaagctaatttgtagccttaagaagagctacagctttcagacaagggcaggactcttggggagaggtggtggtggtgttgctgctagtggtttgttttttgggggagagacgtgggatagagaatctcttctcaccagcatcaccaccacctcatacagagcctatgttgaaatagaaaaaagtggcctttttctggaggatgcagcccctaccaggaagtaactgcttggctagcaaagtgcaagccggagtcctgcccccattttccttgtt

SEQ ID NO: 11 - DNA, 7855 bp, synthetic construct
  source 1..7855: mol_type = other DNA
ggagttgaagactagcctgagcaacatggcaagatcctgtctcaaaattctttttaaaatttttttaaaagcccaggcttggtggcgcatgcctatagttccagctactcaggaggctgaggcagaaggatctcttgaacccaggagtttcaggctgtagttcactatgatggcagctgtgaatagcctgggcaacacagcaagacctcatctccaaaaaaagacaaaagaactaaattattctactgcagaacatgattaggtaaatatctccaaagcagaaagacaggtttcatattttcgttagtttgagtcagtccttccaaatcaaatcttgttttttattagtatacagatggtatagccagtaagtaaatgagaagcagtctttttaagccgatccattcttaaatgaaaaaatatataaatattttagaataaatttattaaattctaaagttgtagaatttttaaatttggatattttgggaaaatatttaaaccactattgcaaacaaaacaacaaaatgtacttatgtttatacttaggcacaaagaaaactacagtattttaaagtaaccattacacaatattgaggttgcaaagattactgaaggcataacctaaaaaatgagttgatttctaaaaatgggaaaaaggaaaaaaataatttctaaaaacaagtatgcatacctaaacctacctaatgacaccttagaaaattcaagtatagcaccattcattaacatcaatgaggatgtcatcacacatcatgtagcctctgcacaccgtgagaataaatgaaaaagacaggcatcttgctatcatgacaatagttttgacctcgcagacctctctgtgcttacgcaacggataaagccataagaactgtcctgccctcaaggagcaacctaaagtaggaaaaaaaaaacaaaattacacaattattatttacaattgtgagaagagctcttgacaacattcaaatggaggatacagtgtagtaagggtgagggtatcaaggcttaattgagaagtgatgttttgaggccatggcttgtcgacgacggcgacctcagtggtgtacggtacaaacccagagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagcttggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagccaccatgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaaagctgtagaggaaccccttaatgcattcaaagaatcaaaaggaatgatgaatgatgaataacaagtttgtacaaaaaagcaggctcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagtgcgggtgccagggcgtgcccccgggctccccgggcgcgtactccgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcacccagctttcttgtacaaagtgggccaccatgagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacgccaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtgccccaactggggtaacctccgagttctctcagttggggggagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagctttgggtttgtaccgtacaccactgagcacgccgtcgtcgacaagcctctttcttctcaataactggtatttcattcctgaatcctttaacttccttaccattgtcactcctaagccaaatctcattacgtcatgtctagactactgttaagagaaccacttaagtggtctctgcagccctcaatttattggtgttatctatgggaaaattgctcaaactctaagccttagtttcctaccctataaaatggggttttatatacaaggaacatactaaatacacaggtatacctcagaaacacggcaggctcaattccagagcactacaataaagcgaatctcatgaatttgttggtttcccagtgcataaattatggttacactataccatagtctattaagaagtgtacaatagcattatgtataattgataaatacatcactgctaaaaaaatgctaacaatctttttgctggtggagggtcttacgccaacgttaacgatggctactgactcatcagggtggtggtggttgaagattacgatagctgtggcaatttcttaaaagacaatgaagtttgccacactgacatcctttcacaagacttctctgtagcatgtgatgctgtttgatagcatgatagcattttacccacagtagaacttttttttccttttcctttttttttttttttaaacgcaaggtctcactctgtcacccaggctggagtgcaggggcgccatctcggcttactgcaacctcctcctccctggttcaagagattctcctgcctcagcttcccaagtagctgggactacaggtgtgcaccacacctggctaatttggtagaggtggggtttcaccatgttggcaaggctggtcttgaactcctgacctcaaatgatctaccagtctcggcctcctaaagtactgggattgcaggtgtgagccaccacacccagccagtggaacttatttcaaaattgaagtcaactctctcacaccctggcactgctttatcaaccaggtttctgtaattcctaaatcctttattggcattttaacaatgttcacagcaacttcaccag

SEQ ID NO: 12 - DNA, 5120 bp, synthetic construct
  source 1..5120: mol_type = other DNA
ggagttgaagactagcctgagcaacatggcaagatcctgtctcaaaattctttttaaaatttttttaaaagcccaggcttggtggcgcatgcctatagttccagctactcaggaggctgaggcagaaggatctcttgaacccaggagtttcaggctgtagttcactatgatggcagctgtgaatagcctgggcaacacagcaagacctcatctccaaaaaaagacaaaagaactaaattattctactgcagaacatgattaggtaaatatctccaaagcagaaagacaggtttcatattttcgttagtttgagtcagtccttccaaatcaaatcttgttttttattagtatacagatggtatagccagtaagtaaatgagaagcagtctttttaagccgatccattcttaaatgaaaaaatatataaatattttagaataaatttattaaattctaaagttgtagaatttttaaatttggatattttgggaaaatatttaaaccactattgcaaacaaaacaacaaaatgtacttatgtttatacttaggcacaaagaaaactacagtattttaaagtaaccattacacaatattgaggttgcaaagattactgaaggcataacctaaaaaatgagttgatttctaaaaatgggaaaaaggaaaaaaataatttctaaaaacaagtatgcatacctaaacctacctaatgacaccttagaaaattcaagtatagcaccattcattaacatcaatgaggatgtcatcacacatcatgtagcctctgcacaccgtgagaataaatgaaaaagacaggcatcttgctatcatgacaatagttttgacctcgcagacctctctgtgcttacgcaacggataaagccataagaactgtcctgccctcaaggagcaacctaaagtaggaaaaaaaaaacaaaattacacaattattatttacaattgtgagaagagctcttgacaacattcaaatggaggatacagtgtagtaagggtgagggtatcaaggcttaattgagaagtgatgttttgaggccatggcttgtcgacgacggcgacctcagtggtgtacggtacaaacccagagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagcttggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagccaccatgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaaagctgtagaggaaccccttaatgcattcaaagaatcaaaaggaatgatgaatgatgaataacaagtttgtacaaaaaagcaggctcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagtgcgggtgccagggcgtgcccccgagttctctcagttggggggagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagctttgggtttgtaccgtacaccactgagcacgccgtcgtcgacaagcctctttcttctcaataactggtatttcattcctgaatcctttaacttccttaccattgtcactcctaagccaaatctcattacgtcatgtctagactactgttaagagaaccacttaagtggtctctgcagccctcaatttattggtgttatctatgggaaaattgctcaaactctaagccttagtttcctaccctataaaatggggttttatatacaaggaacatactaaatacacaggtatacctcagaaacacggcaggctcaattccagagcactacaataaagcgaatctcatgaatttgttggtttcccagtgcataaattatggttacactataccatagtctattaagaagtgtacaatagcattatgtataattgataaatacatcactgctaaaaaaatgctaacaatctttttgctggtggagggtcttacgccaacgttaacgatggctactgactcatcagggtggtggtggttgaagattacgatagctgtggcaatttcttaaaagacaatgaagtttgccacactgacatcctttcacaagacttctctgtagcatgtgatgctgtttgatagcatgatagcattttacccacagtagaacttttttttccttttcctttttttttttttttaaacgcaaggtctcactctgtcacccaggctggagtgcaggggcgccatctcggcttactgcaacctcctcctccctggttcaagagattctcctgcctcagcttcccaagtagctgggactacaggtgtgcaccacacctggctaatttggtagaggtggggtttcaccatgttggcaaggctggtcttgaactcctgacctcaaatgatctaccagtctcggcctcctaaagtactgggattgcaggtgtgagccaccacacccagccagtggaacttatttcaaaattgaagtcaactctctcacaccctggcactgctttatcaaccaggtttctgtaattcctaaatcctttattggcattttaacaatgttcacagcaacttcaccag

SEQ ID NO: 13 - DNA, 11685 bp, synthetic construct
  source 1..11685: mol_type = other DNA
ctacggctggcccagatccttccctgccgcctccttcaggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtggggccactagggacaggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccatactgcgctaccctctcccagaacctcgcgaggttcaactatcggaccaatgcgtagagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagcttgtggtttgtctggtcaaccaccgcgttctccgtcgtcaggatcatggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagccaccatgtcacgactggacaagagcaaagtcataaacggcgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccgggggaagcggagccacgaacttctctctgttaaagcaagcaggagatgttgaagaaaaccccgggcctgtggtttgtctggtcaaccaccgcgccctcagtggtgtacggtacaaacccattgtgcgggtgccagggcgtgcccccgggctccccgggcgcgtactccttatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgagtgccccaactggggtaacctccgagttctctcagttgggggaataatagctaactagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtacaagtttgtacaaaaaagcaggctggtaccaaggacagggaagggagcagtggttcacgcctgtaatcccagcaatttgggaggccaaggtgggtagatcacctgagattaggagttggagaccagcctggccaatatggtgaaaccccgtctctaccaaaaaaacaaaaattagctgagcctggtcatgcatgcctggaatcccaacaactcgggaggctgaggcaggagaatcgcttgaacccaggaggcggagattgcagtgagccaagattgtgccactgcactccagcttggttcccaatagaccccgcaggccctacaggttgtcttcccaacttgccccttgctccataccacccccctccaccccataatattatagaaggacacctagtcagacaaaatgatgcaacttaattttattaggacaaggctggtgggcactggagtggcaacttccagggccaggagaggcactggggaggggtcacagggatgccacccttacacctcgttctcgtagcagaagaatctggtggcttcagggaagtccgtggcacagctgatgaagctgtcaatgctggacatgctccgcatgtcgatgacgccctccgtgcgtgcgggcagaggggccacggggctgggcatgctgctcatctccagctcctctggaggcttgctctgcggggccatctccttggtgttgaggatgggctgggactgcttgtacagctcgtccatgccgccggtggagtggcggccctcggagcgttcgtactgttccaccacggtgtagtcctcgttgtgggaggtgatgtccaacttgcggtccacgttgtaggcgccgggcatctgcacgggcttcttggccttgtaggtggtcttgaagtccgccaggtagcgcccgccgtccttcaggcgcagggccatcttaatgtcgcccttcagcacgccgtcctcggggtacaaccgctcggtggacgcttcccagcccattgtcttcttctgcattacggggccgtcaggagggaagttggtgccgcggagcttcaccttgtagatcagggtgccgtcctccagggaggtgtcctgggtcacggtcacggcgccgccgtcctcgaagttcatcacgcggtcccacttgaagccctcggggaaggactgcttatagtagtcggggatgtcggcggggtgcttggtgaaggccctggagccgtacatgaactgaggggacaggatgtcccaggagaagggcagggggccacccttggtcaccttcagcttggcggtctgggtgccctcgtaggggcggccctcgccctcgccctcgatctcgaactcgtggccgttcatggagccctccatgtgcaccttgaaccgcatgaactccttgatcactgcctcgcccttgctcaccacgttgatatcgatctggtccagggggatgtactcgccctcgctggtgatcctgctcttggcggccgctccgctagcgtaggggtcgtcttcttcaacgggcatcatcacggtggcgccatcggcgccagcggcggtcaaggttttctgattctcctcgtcgatctcctgtctctgccttttcagctcatcccaggcgtagtcctcgatgtgcagtctgcacctcagcatgaacacgccgaaaatggtcttgcaggtaatgtctaggaaggcgtgaatgatagcaaaggcctcctcgctgatcacgccggtggtgtgaaagcccagcagccacagcacggggaacatgggccagaagccgaagaagatgaacatggccctctccaggtccttcttggcgcagtcgggcacgtacctgatcctctttctcacctcgttgaaaaagaagtagaagaaaatgctaaacagggtgcagccgatagcgaagtagatgtacttctcggggctcctcacgatgaagctggcgagggcgatcaccagcagcacggcggtgctgccggtgaaggccagcttgtagggcacgtccacggtgaagcacaggtccatcacgatcagggggcatgtcacgatgtagtccaggtaccagtacctgttcaggctaaactcgctggtgctgtccctagcaaagtcgctggtcttggccacgtacatcaggccggttaagaaggcgatggcggcagacagggtattcagttccaggggtcttctcttgggatgggggtgggctccaaagccggttcccctccaagagtagtaccagaacaccacgccgcttccaagcagaatcacggcgctaaatccgtcgatggttgcaagcagggtgtaattcatgctcatctgaggagaaggggcagcctgagcacaagaggtggtgcctccgttaggattggggctttgatctgtgggggccaccacggtagggggtgtaggacaggcgctcatggtggcctgcgctctcaggcacgacacgactcctccgctgcccaccgcagactgaggcagcgctgagtcgccggcgccgcagcgcagatggtcgcgcccgtgcccccctatctcgcgcctcgcgtggtgcggtccggctgggccggcggcggcgcggacgcgaccaaggtggccgggaaggggagtttgcgggggaccggcgagtgacgtcagcgcgccttcagtgctgaggcggcggtggcgcgcgccgccaggcgggggcgaaggcactgtccgcggtgctgaagctggcagtgcgcacgcgcctcgccgcatcctgtttcccctccccctctctgataggggatgcgcaatttggggaatgggggttgggtgcttgtccagtgggtcggggtcggtcgtcaggtaggcacccccaccccgcctcatcctggtcctaaaacccacttgcactcatacgcagggccctctgcagtgggtttgtaccgtacaccactgaggacgcggtggttgaccagacaaaccacggtaccgagctcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgacctatataagcagagctcgtttagtgaaccgtcagatcgcctggagacgccatccacgctgttttgacctccatagaagacaccgggaccgatccagcctccgcggccccgaattgacccagctttcttgtacaaagtgggccaccatggaggcgctaatggcccggggcgcgctcacgggtcccctgagggcgctctgtctcctgggctgcctgctgagccacgccgccgccgcgccgtcgcccatcatcaagttccccggcgatgtcgcccccaaaacggacaaagagttggcagtgcaatacctgaacaccttctatggctgccccaaggagagctgcaacctgtttgtgctgaaggacacactaaagaagatgcagaagttctttggactgccccagacaggtgatcttgaccagaataccatcgagaccatgcggaagccacgctgcggcggcccagatgtggccaactacaacttcttccctcgcaagcccaagtgggacaagaaccagatcacatacaggatcattggctacacacctgatctggacccagagacagtggatgatgcctttgctcgtgccttccaagtctggagcgatgtgaccccactgcggttttctcgaatccatgatggagaggcagacatcatgatcaactttggccgctgggagcatggcgatggatacccctttgacggtaaggacggactcctggctcatgccttcgccccaggcactggtgttgggggagactcccattttgatgacgatgagctatggaccttgggagaaggccaagtggtccgtgtgaagtatgggaacgccgatggggagtactgcaagttccccttcttgttcaatggcaaggagtacaacagctgcactgataccggccgcagcgatggcttcctctggtgctccaccacctacaactttgagaaggatggcaagtacggcttctgtccccatgaagccctgttcaccatgggcggcaacgctgaaggacagccctgcaagtttccattccgcttccagggcacatcctatgacagctgcaccactgagggccgcacggatggctaccgctggtgcggcaccactgaggactacgaccgcgacaagaagtatggcttctgccctgagaccgccatgtccactgttggtgggaactcagaaggtgccccctgtgtcttccccttcactttcctgggcaacaaatatgagagctgcaccagcgccggccgcagtgacggaaagatgtggtgtgcgaccacagccaactacgatgatgaccgcaagtggggcttctgccctgaccaagggtacagcctgttcctcgtggcagcccacgagtttggccacgccatggggctggagcactcccaagaccctggggccctgatggcacccatttacacctacaccaagaacttccgtctgtcccaggatgacatcaagggcattcaggagctctatggggcctctcctgacattgaccttggcaccggccccacccccacgctgggccctgtcactcctgagatctgcaaacaggacattgtatttgatggcatcgctcagatccgtggtgagatcttcttcttcaaggaccggttcatttggcggactgtgacgccacgtgacaagcccatggggcccctgctggtggccacattctggcctgagctcccggaaaagattgatgcggtatacgaggccccacaggaggagaaggctgtgttctttgcagggaatgaatactggatctactcagccagcaccctggagcgagggtaccccaagccactgaccagcctgggactgccccctgatgtccagcgagtggatgccgcctttaactggagcaaaaacaagaagacatacatctttgctggagacaaattctggagatacaatgaggtgaagaagaaaatggatcctggcttccccaagctcatcgcagatgcctggaatgccatccccgataacctggatgccgtcgtggacctgcagggcggcggtcacagctacttcttcaagggtgcctattacctgaagctggagaaccaaagtctgaagagcgtgaagtttggaagcatcaaatccgactggctaggctgctgatcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcatgatcctgacgacggagctcgcggtggttgaccagacaaaccacgagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagcttcaacgataggtgccgaccttatgtcgcgtagagctgctctgacgcggccgtctattgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttctctttctccatccttctttccttaaagagtccccagtgctatctgggacatattcctccgcccagagcagggtcccgcttccctaaggccctgctctgggcttctgggtttgagtccttggcaagcccaggagaggcgctcaggcttccctgtcccccttcctcgtccaccatctcatgcccctggctctcctgccccttccctacaggggttcctggctctgctcttcagactgagccccgttcccctgcatccccgttcccctgcatcccccttcccctgcatcccccagaggccccaggccacctacttggcctggaccccacgagaggccaccccagccctgtctaccaggctgccttttgggtggattctcctccaactgtggggtgactgcttggcaaactcactcttcggggtatcccaggaggcctggagcattggggtgggctggggttcagagaggagggattcccttctcaggttacgtggccaagaagcaggggagctgggtttgggtcaggtctgggtgtggggtgaccagcttatgctgtttgcccaggacagcctagttttagcactgaaaccctcagtcctaggaaaacagggatggttggtcactgtctctgggtgactcttgattcccggccagtttctccacctggggctgtgtttctcgtcctgcatccttctccaggcagg

SEQ ID NO: 14 - DNA, 10918 bp, synthetic construct
  source 1..10918: mol_type = other DNA
ctacggctggcccagatccttccctgccgcctccttcaggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtggggccactagggacaggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccatactgcgctaccctctcccagaacctcgcgaggttcaactatcggaccaatgcgtagagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagcttgtggtttgtctggtcaaccaccgcgttctccgtcgtcaggatcatggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagccaccatgtcacgactggacaagagcaaagtcataaacggcgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccgggggaagcggagccacgaacttctctctgttaaagcaagcaggagatgttgaagaaaaccccgggcctgtggtttgtctggtcaaccaccgcgccctcagtggtgtacggtacaaacccattgtgcgggtgccagggcgtgcccccgagttctctcagttgggggaataatagctaactagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtacaagtttgtacaaaaaagcaggctggtaccaaggacagggaagggagcagtggttcacgcctgtaatcccagcaatttgggaggccaaggtgggtagatcacctgagattaggagttggagaccagcctggccaatatggtgaaaccccgtctctaccaaaaaaacaaaaattagctgagcctggtcatgcatgcctggaatcccaacaactcgggaggctgaggcaggagaatcgcttgaacccaggaggcggagattgcagtgagccaagattgtgccactgcactccagcttggttcccaatagaccccgcaggccctacaggttgtcttcccaacttgccccttgctccataccacccccctccaccccataatattatagaaggacacctagtcagacaaaatgatgcaacttaattttattaggacaaggctggtgggcactggagtggcaacttccagggccaggagaggcactggggaggggtcacagggatgccacccttacacctcgttctcgtagcagaagaatctggtggcttcagggaagtccgtggcacagctgatgaagctgtcaatgctggacatgctccgcatgtcgatgacgccctccgtgcgtgcgggcagaggggccacggggctgggcatgctgctcatctccagctcctctggaggcttgctctgcggggccatctccttggtgttgaggatgggctgggactgcttgtacagctcgtccatgccgccggtggagtggcggccctcggagcgttcgtactgttccaccacggtgtagtcctcgttgtgggaggtgatgtccaacttgcggtccacgttgtaggcgccgggcatctgcacgggcttcttggccttgtaggtggtcttgaagtccgccaggtagcgcccgccgtccttcaggcgcagggccatcttaatgtcgcccttcagcacgccgtcctcggggtacaaccgctcggtggacgcttcccagcccattgtcttcttctgcattacggggccgtcaggagggaagttggtgccgcggagcttcaccttgtagatcagggtgccgtcctccagggaggtgtcctgggtcacggtcacggcgccgccgtcctcgaagttcatcacgcggtcccacttgaagccctcggggaaggactgcttatagtagtcggggatgtcggcggggtgcttggtgaaggccctggagccgtacatgaactgaggggacaggatgtcccaggagaagggcagggggccacccttggtcaccttcagcttggcggtctgggtgccctcgtaggggcggccctcgccctcgccctcgatctcgaactcgtggccgttcatggagccctccatgtgcaccttgaaccgcatgaactccttgatcactgcctcgcccttgctcaccacgttgatatcgatctggtccagggggatgtactcgccctcgctggtgatcctgctcttggcggccgctccgctagcgtaggggtcgtcttcttcaacgggcatcatcacggtggcgccatcggcgccagcggcggtcaaggttttctgattctcctcgtcgatctcctgtctctgccttttcagctcatcccaggcgtagtcctcgatgtgcagtctgcacctcagcatgaacacgccgaaaatggtcttgcaggtaatgtctaggaaggcgtgaatgatagcaaaggcctcctcgctgatcacgccggtggtgtgaaagcccagcagccacagcacggggaacatgggccagaagccgaagaagatgaacatggccctctccaggtccttcttggcgcagtcgggcacgtacctgatcctctttctcacctcgttgaaaaagaagtagaagaaaatgctaaacagggtgcagccgatagcgaagtagatgtacttctcggggctcctcacgatgaagctggcgagggcgatcaccagcagcacggcggtgctgccggtgaaggccagcttgtagggcacgtccacggtgaagcacaggtccatcacgatcagggggcatgtcacgatgtagtccaggtaccagtacctgttcaggctaaactcgctggtgctgtccctagcaaagtcgctggtcttggccacgtacatcaggccggttaagaaggcgatggcggcagacagggtattcagttccaggggtcttctcttgggatgggggtgggctccaaagccggttcccctccaagagtagtaccagaacaccacgccgcttccaagcagaatcacggcgctaaatccgtcgatggttgcaagcagggtgtaattcatgctcatctgaggagaaggggcagcctgagcacaagaggtggtgcctccgttaggattggggctttgatctgtgggggccaccacggtagggggtgtaggacaggcgctcatggtggcctgcgctctcaggcacgacacgactcctccgctgcccaccgcagactgaggcagcgctgagtcgccggcgccgcagcgcagatggtcgcgcccgtgcccccctatctcgcgcctcgcgtggtgcggtccggctgggccggcggcggcgcggacgcgaccaaggtggccgggaaggggagtttgcgggggaccggcgagtgacgtcagcgcgccttcagtgctgaggcggcggtggcgcgcgccgccaggcgggggcgaaggcactgtccgcggtgctgaagctggcagtgcgcacgcgcctcgccgcatcctgtttcccctccccctctctgataggggatgcgcaatttggggaatgggggttgggtgcttgtccagtgggtcggggtcggtcgtcaggtaggcacccccaccccgcctcatcctggtcctaaaacccacttgcactcatacgcagggccctctgcagtgggtttgtaccgtacaccactgaggacgcggtggttgaccagacaaaccacggtaccgagctcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgacctatataagcagagctcgtttagtgaaccgtcagatcgcctggagacgccatccacgctgttttgacctccatagaagacaccgggaccgatccagcctccgcggccccgaattgacccagctttcttgtacaaagtgggccaccatggaggcgctaatggcccggggcgcgctcacgggtcccctgagggcgctctgtctcctgggctgcctgctgagccacgccgccgccgcgccgtcgcccatcatcaagttccccggcgatgtcgcccccaaaacggacaaagagttggcagtgcaatacctgaacaccttctatggctgccccaaggagagctgcaacctgtttgtgctgaaggacacactaaagaagatgcagaagttctttggactgccccagacaggtgatcttgaccagaataccatcgagaccatgcggaagccacgctgcggcggcccagatgtggccaactacaacttcttccctcgcaagcccaagtgggacaagaaccagatcacatacaggatcattggctacacacctgatctggacccagagacagtggatgatgcctttgctcgtgccttccaagtctggagcgatgtgaccccactgcggttttctcgaatccatgatggagaggcagacatcatgatcaactttggccgctgggagcatggcgatggatacccctttgacggtaaggacggactcctggctcatgccttcgccccaggcactggtgttgggggagactcccattttgatgacgatgagctatggaccttgggagaaggccaagtggtccgtgtgaagtatgggaacgccgatggggagtactgcaagttccccttcttgttcaatggcaaggagtacaacagctgcactgataccggccgcagcgatggcttcctctggtgctccaccacctacaactttgagaaggatggcaagtacggcttctgtccccatgaagccctgttcaccatgggcggcaacgctgaaggacagccctgcaagtttccattccgcttccagggcacatcctatgacagctgcaccactgagggccgcacggatggctaccgctggtgcggcaccactgaggactacgaccgcgacaagaagtatggcttctgccctgagaccgccatgtccactgttggtgggaactcagaaggtgccccctgtgtcttccccttcactttcctgggcaacaaatatgagagctgcaccagcgccggccgcagtgacggaaagatgtggtgtgcgaccacagccaactacgatgatgaccgcaagtggggcttctgccctgaccaagggtacagcctgttcctcgtggcagcccacgagtttggccacgccatggggctggagcactcccaagaccctggggccctgatggcacccatttacacctacaccaagaacttccgtctgtcccaggatgacatcaagggcattcaggagctctatggggcctctcctgacattgaccttggcaccggccccacccccacgctgggccctgtcactcctgagatctgcaaacaggacattgtatttgatggcatcgctcagatccgtggtgagatcttcttcttcaaggaccggttcatttggcggactgtgacgccacgtgacaagcccatggggcccctgctggtggccacattctggcctgagctcccggaaaagattgatgcggtatacgaggccccacaggaggagaaggctgtgttctttgcagggaatgaatactggatctactcagccagcaccctggagcgagggtaccccaagccactgaccagcctgggactgccccctgatgtccagcgagtggatgccgcctttaactggagcaaaaacaagaagacatacatctttgctggagacaaattctggagatacaatgaggtgaagaagaaaatggatcctggcttccccaagctcatcgcagatgcctggaatgccatccccgataacctggatgccgtcgtggacctgcagggcggcggtcacagctacttcttcaagggtgcctattacctgaagctggagaaccaaagtctgaagagcgtgaagtttggaagcatcaaatccgactggctaggctgctgatcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcatgatcctgacgacggagctcgcggtggttgaccagacaaaccacgagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagcttcaacgataggtgccgaccttatgtcgcgtagagctgctctgacgcggccgtctattgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttctctttctccatccttctttccttaaagagtccccagtgctatctgggacatattcctccgcccagagcagggtcccgcttccctaaggccctgctctgggcttctgggtttgagtccttggcaagcccaggagaggcgctcaggcttccctgtcccccttcctcgtccaccatctcatgcccctggctctcctgccccttccctacaggggttcctggctctgctcttcagactgagccccgttcccctgcatccccgttcccctgcatcccccttcccctgcatcccccagaggccccaggccacctacttggcctggaccccacgagaggccaccccagccctgtctaccaggctgccttttgggtggattctcctccaactgtggggtgactgcttggcaaactcactcttcggggtatcccaggaggcctggagcattggggtgggctggggttcagagaggagggattcccttctcaggttacgtggccaagaagcaggggagctgggtttgggtcaggtctgggtgtggggtgaccagcttatgctgtttgcccaggacagcctagttttagcactgaaaccctcagtcctaggaaaacagggatggttggtcactgtctctgggtgactcttgattcccggccagtttctccacctggggctgtgtttctcgtcctgcatccttctccaggcagg

SEQ ID NO: 15 - DNA, 7673 bp, synthetic construct
  source 1..7673: mol_type = other DNA
catgtatagaggaattatgagggaaagataccaagtcacggtttattcttcaaaatggaggtggcttgttgggaaggtggaagctcatttggccagagtggaaatggaattgggagaaatcgatgaccaaatgtaaacacttggtgcctgatatagcttgacaccaagttagccccaagtgaaataccctggcaatattaatgtgtcttttcccgatattcctcaggtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtggggtaagtcttacattcttttgtaagctgctgaaagttgtgtatgagtagtcatatcataaagctgctttgatataaaaaaggtctatggccatactaccctgaatgagtcccatcccatctgatataaacaatctgcatattgggattgtcagggaatgttcttaaagatcagattagtggcacctgctgagatactgatgcacagcatggtttctgaaccagtagtttccctgcagttgagcagggagcagcagcagcacttgcacaaatacatatacactcttaacacttcttacctactggcttcctctagcttttgtggcagcttcaggtatatttagcactgaacgaacatctcaagaaggtataggcctttgtttgtaagtcctgctgtcctagcatcctataatcctggacttctccagtactttctggctggattggtatctgaggctagtaggaagggcttgttcctgctgggtagctctaaacaatgtattcatgggtaggaacagcagcctattctgccagccttatttctaaccattttagacatttgttagtacatggtattttaaaagtaaaacttaatgtcttccttttttttctccactgtctttttcatagatcgagacatgggaggcggtgggtctggtggcggaggctcgggcggaggtgggtcgggtggcggcggatcaatggtagatggaaccctccttttactcctctcggaggccctggcccttacccagacctgggcgggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgggaagcggagagggcaggggaagtcttctaacatgcggggacgtggaggaaaatcccggccccatgcccacgctactgcgggtttatatagacggtcctcacgggatggggaaaaccaccaccacgcaactgctggtggccctgggttcgcgcgacgatatcgtctacgtacccgagccgatgacttactggcaggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtacaacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccacccccggctccataccgacgatctgcgacctggcgcgcacgtttgcccgggagatgggggaggctaactgagtgcgggtgccagggcgtgcccccgggctccccgggcgcgtactcccaagtttgtacaaaaaagcaggctggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggaagcggagagggcaggggaagtcttctaacatgcggggacgtggaggaaaatcccggccccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaacccagctttcttgtacaaagtgggtgccccaactggggtaacctccgagttctctcagttggggggcagcatcatggaggtaagtttttgaccttgagaaaatgtttttgtttcactgtcctgaggactatttatagacagctctaacatgataaccctcactatgtggagaacattgacagagtaacattttagcagggaaagaagaatcctacagggtcatgttcccttctcctgtggagtggcatgaagaaggtgtatggccccaggtatggccatattactgaccctctacagagagggcaaaggaactgccagtatggtattgcaggataaaggcaggtggttacccacattacctgcaaggctttgatctttcttctgccatttccacattggacatctctgctgaggagagaaaatgaaccactcttttcctttgtataatgttgttttattcttcagacagaagagaggagttatacagctctgcagacatcccattcctgtatggggactgtgtttgcctcttagaggttcccaggccactagaggagataaagggaaacagattgttataacttgatataatgatactataatagatgtaactacaaggagctccagaagcaagagagagggaggaacttggacttctctgcatctttagttggagtccaaaggcttttcaatgaaattctactgcccagggtacattgatgctgaaaccccattcaaatctcctgttatattctagaacagggaattgatttgggagagcatcaggaaggtggatgatctgcccagtcacactgttagtaaattgtagagccaggacctgaactctaatatagtcatgtgttacttaatgacggggacatgttctgagaaatgcttacacaaacctaggtgttgtagcctactacacgcataggctacatggtatagcctattgctcctagactacaaacctgtacagcctgttactgtactgaatactgtgggcagttgtaacacaatggtaagtatttgtgtatctaaacatagaagttgcagtaaaaatatgctattttaatcttatgagaccactgtcatatatacagtccatcattgaccaaaacatcatatcagcattttttcttctaagattttgggagcaccaaagggatacactaacaggatatactctttataatgggtttggagaactgtctgcagctacttcttttaaaaaggtgatctacacagtagaaattagacaagtttggtaatgagatctgcaatccaaataaaataaattcattgctaacctttttcttttcttttcaggtttgaagatgccgcatttggattggatgaattccaaattctgcttgcttgctttttaatattgatatgcttatacacttacactttatgcacaaaatgtagggttataataatgttaacatggacatgatcttctttataattctactt

SEQ ID NO: 16 - DNA, 4793 bp, synthetic construct; identical to SEQ ID NO: 2
  source 1..4793: mol_type = other DNA

SEQ ID NO: 17 - DNA, 933 bp, Wobblia lunata
  source 1..933: mol_type = other DNA
atgagcgcctgccccaccccccccaccgtggtggcccccaccgaccagagccccaaccccaacggcggcaccaccagctgcgcccaggccgcccccagcccccagatgagcatgaactacaccctgctggccaccatcgacggcttcagcgccgtgatcctgctgggcagcggcgtggtgttctggtactacagctggcgcggcaccggcttcggcgcccacccccaccccaagcgccgccccctggagctgaacaccctgagcgccgccatcgccttcctgaccggcctgatgtacgtggccaagaccagcgacttcgcccgcgacagcaccagcgagttcagcctgaaccgctactggtacagcgactacatcgtgacctgccccctgatcgtgatggacctgtgcttcaccgtggacgtgccctacaagctggccttcaccggcagcaccgccgtgctgctggtgatcgccctggccagcttcatcgtgcgcagccccgagaagtacatctacttcgccatcggctgcaccctgttcagcatcttcttctacttcttcttcaacgaggtgcgcaagcgcatccgctacgtgcccgactgcgccaagaaggacctggagcgcgccatgttcatcttcttcggcttctggcccatgttccccgtgctgtggctgctgggcttccacaccaccggcgtgatcagcgaggaggccttcttcatcatccacgccttcctggacatcacctgcaagaccatcttcggcgtgttcatgctgcgctgccgcctgcacatcgaggactacgcctgggacgagctgaagcgccagcgccaggagatcgacgaggagaaccagaagaccctgaccgccgccggcgccgacggcgccaccgtgatgatgcccgtggaggaggacgacccctacgccagcgga

(19) United States
(12) Patent Application Publication
(10) Pub. No.: US 2026/0022335 A1
(43) Pub. Date: Jan. 22, 2026

(54) A HYPOIMMUNOGENIC CELL AND METHODS OF GENERATION THEREOF

(71) Applicant: Science Corporation, Alameda, CA (US)

(72) Inventors: Alan Mardinly, Alameda, CA (US); Kevin Smith, Alameda, CA (US); Satoru Miura, Alameda, CA (US)

(73) Assignee: Science Corporation, Alameda, CA (US)

(21) Appl. No.: 19/265,825

(22) Filed: Jul. 10, 2025

Related U.S. Application Data
(63) Continuation-in-part of application No. 18/660,713, filed on May 10, 2024, now abandoned.
(60) Provisional application No. 63/465,453, filed on May 10, 2023.

(51) Int. Cl. C12N 5/079; A61L 27/36; C07K 14/47; C07K 14/74

(52) U.S. Cl. CPC C12N 5/0618; A61L 27/3687; C07K 14/47; C07K 14/70539; C07K 2319/00; C12N 2510/00

(57) ABSTRACT

In variants, the method of generating a hypoimmunogenic cell can include: integrating an insertion sequence into a cell genome (e.g., into a Beta-2-Microglobulin (B2M) gene) and performing a cell selection. The method can optionally include: integrating a selection sequence into the cell genome, removing a selectable marker, editing an additional gene, and/or any other suitable steps. The hypoimmunogenic cell can include a genetically engineered sequence including: a Beta-2-Microglobulin:human leukocyte antigen (B2M:HLA) fusion gene, a multicistronic element, and a kill switch gene.

### CROSS-REFERENCE TO RELATED APPLICATIONS

This application is a continuation-in-part of U.S. application Ser. No. 18/660,713 filed 10 May 2024, which claims the benefit of U.S. Provisional Application No. 63/465,453 filed 10 May 2023, each of which is incorporated in its entirety by this reference.

### INCORPORATION BY REFERENCE OF A SEQUENCE LISTING

A Sequence Listing is provided herewith as a Sequence Listing.xml file. The Sequence Listing, created on 10 Jul. 2025, is identified as “SCIX-P16-US-APP-SEQ-LST.xml” and is 129,442 bytes in size. The content of the Sequence Listing is incorporated in its entirety herein by this reference.

### TECHNICAL FIELD

This invention relates generally to the genetic engineering field, and more specifically to a new and useful hypoimmunogenic cell and methods of generation thereof in the genetic engineering field.

### BRIEF DESCRIPTION OF THE FIGURES

FIG. 1 is a schematic representation of a variant of the method.

FIG. 2A depicts a first example of the method, including integrating a selection sequence into a cell genome (e.g., into a B2M gene), performing a cell selection, and replacing all or a portion of the selection sequence with an insertion sequence.

FIG. 2B depicts a second example of the method, including integrating an insertion sequence into a cell genome (e.g., into a B2M gene), performing a cell selection, and removing a selectable marker.

FIG. 3A depicts an example of replacing a selection sequence with an insertion sequence.

FIG. 3B depicts an example of replacing part of the B2M gene with an insertion sequence.

FIG. 3C depicts an example of inserting an insertion sequence into the B2M gene.

FIG. 4A depicts a first example of a genetically engineered sequence, with a kill switch gene on the 5′ side of a B2M:HLA fusion gene.

FIG. 4B depicts a second example of a genetically engineered sequence, with a kill switch gene on the 3′ side of a B2M:HLA fusion gene.

FIGS. 5A-5C depict specific examples of two alleles of a genetically engineered B2M sequence in a cell.

FIG. 6A depicts a first specific example of a genetically engineered sequence, with a kill switch gene on the 5′ side of a B2M:HLA fusion gene.

FIG. 6B depicts a second specific example of a genetically engineered sequence, with a kill switch gene on the 3′ side of a B2M:HLA fusion gene.

FIGS. 7A-7H depict examples of an edited B2M gene (after selectable marker removal) with an insertion sequence replacing all or part of the B2M gene.

FIGS. 8A-8C depict examples of an edited B2M gene (after selectable marker removal) with an insertion sequence inserted into the B2M gene.

FIGS. 9A-9D depict examples of an edited B2M gene with an insertion sequence integrated into the B2M gene via replacing a selection sequence, wherein the selection sequence replaced all or part of the B2M gene.

FIG. 10 depicts an example of an edited B2M gene with an insertion sequence integrated into the B2M gene via replacing a selection sequence, wherein the selection sequence was inserted into the B2M gene.

FIG. 11A depicts an illustrative example of a vector including an insertion sequence.

FIG. 11B depicts an illustrative example of a vector including a recombinase that functions to excise a selection sequence from the B2M gene.

FIG. 12A depicts a specific example of a control cell (e.g., a control for the cell depicted in FIG. 12B).

FIG. 12B depicts a specific example of cell (e.g., a neuron) with hypoimmunogenic properties.

FIG. 12C depicts another specific example of cell (e.g., a neuron) with hypoimmunogenic properties.

FIG. 12D depicts another specific example of cell (e.g., a neuron) with hypoimmunogenic properties.

### DETAILED DESCRIPTION

The following description of the embodiments of the invention is not intended to limit the invention to these embodiments, but rather to enable any person skilled in the art to make and use this invention.

As used herein, “sequence” preferably refers to a nucleotide sequence, such as a DNA sequence, an RNA sequence, or other sequences of nucleotides, but can also refer to an amino acid sequence, or any other biological sequence of repeating units. Nucleic acids according to embodiments can have any suitable form, including linear, circular, and any suitable strandedness, including single stranded, and double stranded, for any particular host species and/or purpose. Also, nucleic acids according to examples can be incorporated into genetic constructs according to examples, as described in detail below. Also, while DNA sequences are described herein, it is understood that corresponding RNA sequences are contemplated by the inventors and are considered within the scope of the invention. RNA sequences may have individual utility beyond the utility of DNA sequences.

Nucleic acids according to embodiments can be synthesized using any suitable techniques, procedures, and processes, such as solid-phase synthesis, enzymatic synthesis, such as reverse transcription using reverse transcriptase to generate complementary DNA (cDNA) from an RNA template. Polymerase chain reaction (PCR) and polymerase cycling assembly (PCA) techniques can also be used in synthesis of nucleic acids according to embodiments.

Once synthesized, nucleic acids according to embodiments can be further processed and/or treated using one or more suitable techniques, procedures, and processes for purification, quality assessment, and quantification. For example, nucleic acids according to embodiments can be subjected to high-performance liquid chromatography (HPLC), ultrafiltration, size-exclusion chromatography, or other suitable techniques for removing contaminants. Quality and quantity assessments can be conducted using spectroscopy, electrophoresis, or other analytical methods to ensure integrity and consistency of the synthesized nucleic acids according to embodiments.

As used herein, the term “percent identity” refers to a percentage of identity between two referenced nucleotide sequences. The percent identity of two nucleotide sequences is determined by aligning the referenced sequences for optimal comparison (e.g., introducing gaps if necessary). The percent identity is calculated as follows:

% identity=(# of identical positions/total # of positions)×100
where an identical position is one in which the nucleotide in one sequence is the same as the nucleotide in the corresponding position of the other sequence. A non-limiting example of a sequence comparison algorithm is described in Karlin et al., Proc. Natl. Acad. Sci. USA 90:5873-5877 (1993). This algorithm is implemented in the NBLAST and XBLAST programs (version 2.0), as described in Altschul et al., Nucleic Acids Res. 25:389-3402 (1997). Unless otherwise specified, the default parameters of the respective BLAST programs (e.g., NBLAST) may be used for sequence comparison. In some embodiments, parameters may be set at a score threshold of 100 and a word length of 12, with optional variations such as a word length of 5 or 20.

As used herein, the term “plurality” refers to a quantity of at least two members. In some embodiments, a plurality may comprise 5 or more, 10 or more, 50 or more, 100 or more, 1000 or more, 10,000 or more, or up to 100,000 or more members.

Where a range of values is provided, it is understood that each intervening value within the stated range—including values to the nearest tenth of the unit of the lower limit, unless the context clearly dictates otherwise—is encompassed within the described range. The upper and lower limits of the range, as well as any intermediate values, are included within the scope of the disclosure unless explicitly stated otherwise. Additionally, where a range includes one or both of its endpoints, variations excluding either or both endpoints are also encompassed within the disclosure.

Unless otherwise defined, all technical and scientific terms used herein have the same meaning as commonly understood by those of ordinary skill in the relevant art. While methods and systems similar or functionally equivalent to those described herein may be used in the practice or testing of the disclosed methods and systems, representative and illustrative embodiments are described below.

For clarity, certain features of the disclosed systems, methods, and compositions are described separately in distinct examples. However, it is understood that these features may also be combined into a single example. Conversely, features that are described together in a single example may also be provided separately or in any operable sub-combination. All combinations and sub-combinations of the disclosed examples are expressly included within the scope of the present disclosure, provided they result in an operable system, method, or composition.

As will be apparent to those skilled in the art upon reading this disclosure, each of the examples described herein comprises distinct components, features, and/or steps that may be separated or combined without departing from the scope or spirit of the invention. Any described method may be carried out in the order of steps explicitly recited or in any logically permissible order unless otherwise stated.

### 1. Overview

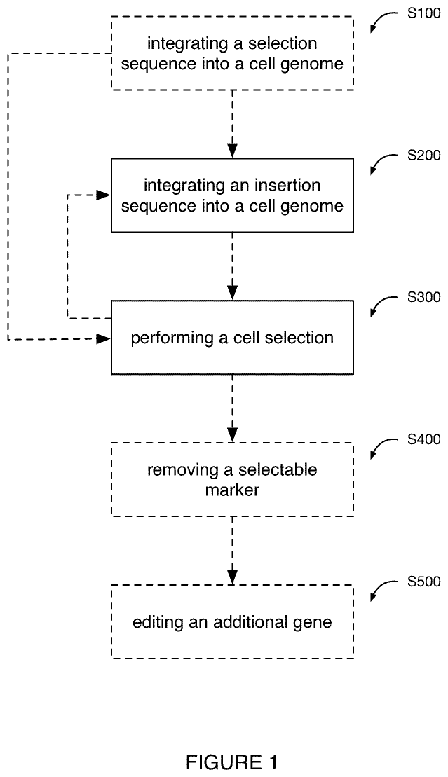

As shown in FIG. 1, the method can include: integrating an insertion sequence into a cell genome S200 and performing a cell selection S300. However, the method can additionally or alternatively include any other suitable steps. In variants, the method can function to create a hypoimmunogenic cell line containing a kill switch gene (e.g., co-transcribed with a histocompatibility antigen gene) to induce cell death as desired or needed. The safeguarded hypoimmunogenic cell line can then be used as a template cell line that can be further edited (e.g., to provide one or more target functionalities) and safely implanted in a human patient or other organism, or otherwise used. In a specific example, the cell line can be a CIITA knockout cell line with a B2M:HLA fusion protein and a kill switch protein, wherein the kill switch gene is in the same open reading frame with the B2M-HLA fusion gene.

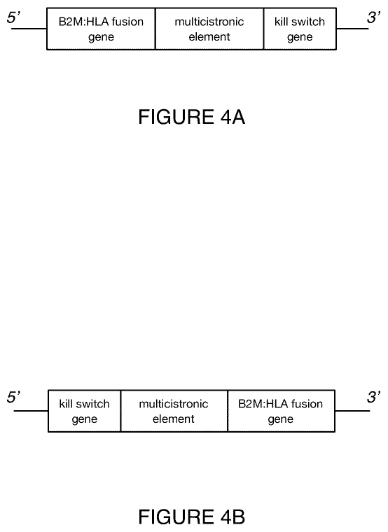

As shown in FIG. 4A and FIG. 4B, a genetically engineered sequence can include: a Beta-2-Microglobulin (B2M): human leukocyte antigen (HLA) fusion gene; a multicistronic element; and a kill switch gene. However, the genetically engineered sequence can additionally or alternatively include any other suitable sequences. In variants, a cell containing the genetically engineered sequence can function as a safeguarded hypoimmunogenic cell.

### 2. Examples

In an example, the method includes integrating an insertion sequence into the Beta-2-Microglobulin (B2M) gene of a cell, wherein the insertion sequence includes a human leukocyte antigen (HLA) class I gene (e.g., HLA-E, HLA-G, etc.), a multicistronic element (e.g., a cleavage site), a kill switch gene, an optional linker, one or more optional B2M replacement sequences, and an optional selection sequence (e.g., selectable markers and a promoter bounded by recombination sites). In examples, the insertion sequence can be integrated into the B2M gene (e.g., fused to the B2M gene) by: replacing all or a portion of the B2M gene with the insertion sequence, inserting the insertion sequence into the B2M gene, and/or replacing an initial selection sequence integrated into the B2M gene with the insertion sequence. The method can optionally include editing multiple B2M loci, wherein both B2M alleles in a cell are edited. Once the insertion sequence is integrated, the edited cell genome includes, in the same open reading frame: a B2M:HLA fusion gene (e.g., including all or a portion of the B2M gene, the linker, and the HLA gene), the multicistronic element, and the kill switch gene. The multicistronic element can be positioned between the B2M:HLA fusion gene and the kill switch gene such that the B2M:HLA-E fusion gene and kill switch gene are co-transcribed but generate separate proteins, enabling the kill switch protein to remain inside the cell while the B2M:HLA-E fusion protein travels to the cell surface.

### 3. Technical Advantages

Variants of the technology can confer one or more advantages over conventional technologies.

First, variants of the technology can create a hypoimmunogenic cell (e.g., a hypoimmunogenic engraftable cell) by preventing HLA class II function (e.g., knocking out CIITA to prevent HLA class II expression) and by fusing the B2M gene to an HLA gene (e.g., HLA-E gene, HLA-G gene, etc.) to generate a B2M:HLA fusion protein when the B2M:HLA fusion gene is translated. This can enable the fused linker (e.g., HLA-E in a B2M:HLA-E fusion protein) to remain functional while eliminating free-floating B2M proteins, thus preventing presentation of other HLA class I proteins on the cell surface. Variants of the technology can optionally include editing both B2M alleles in a cell. In a specific example, one B2M allele can be edited to become an B2M:HLA-E fusion gene and one B2M allele can be edited to become a B2M:HLA-G fusion gene. This can increase the hypoimmunogenicity of the cell (e.g., avoiding more immunogenic interactions relative to a cell with only B2M:HLA-E fusion genes or only B2M:HLA-G fusion genes)

Second, independent integration of a kill switch gene into a hypoimmunogenic cell can be fallible (the kill switch gene can be improperly integrated, the cell can drop the kill switch gene edit, etc.), leading to inability to kill the grafted cells. Variants of the technology can edit a cell genome to create a B2M:HLA fusion gene (contributing to cell hypoimmunogenicity) such that a kill switch gene is in the same open reading frame (co-transcribed, co-translated) as the B2M:HLA fusion gene. For example, an insertion sequence containing an HLA gene, a multicistronic element, and a kill switch gene can be integrated into the B2M gene. If the insertion sequence is not properly integrated, the cell will not gain hypoimmunogenicity and thus can be destroyed by the immune system; if the insertion sequence is properly integrated and transcribed/translated, the cell will have a kill switch protein available to provide an avenue to induce cell death. In a specific example, the insertion sequence can include a multicistronic element positioned between the kill switch gene and the B2M:HLA fusion gene such that the kill switch protein is separate from the B2M:HLA protein, enabling the kill switch protein to remain functional within the cell while the B2M:HLA protein travels to the cell surface.

Third, variants of the technology can use selectable markers to verify proper integration of the insertion sequence into the B2M gene. In a first example, the method can include: integrating a selection sequence (including selectable markers) into the B2M gene, performing a positive cell selection to verify the integration, replacing the selection sequence with the insertion sequence, and optionally performing a negative selection to verify the replacement. In a second example, the method can include: integrating the insertion sequence including selectable markers into the B2M gene, performing a positive cell selection to verify the integration, and then excising the selectable markers.

However, further advantages can be provided by the system and method disclosed herein.

### 4. Method

As shown in FIG. 1, the method can include: integrating an insertion sequence into a cell genome S200 and performing a cell selection S300. The method can optionally include integrating a selection sequence into a cell genome S100, removing a selectable marker S400, editing an additional gene S500, and/or any other suitable steps.

The method can optionally include any or all of the processes, embodiments, and examples as described in U.S. application Ser. No. 18/662,102 filed 13 May 2024, titled “MODULAR GENETICALLY ENGINEERED CELL AND METHODS OF GENERATION THEREOF,” claiming priority to U.S. Provisional Application No. 63/465,759 filed 11 May 2023, each of which is incorporated in its entirety by this reference. In a specific example, the method can include integrating one or more landing sites into the cell genome.

All or parts of method can be performed once (e.g., for a single cell), multiple times (e.g., one or more times for each of a set of cells, one or more times for each of a set of a set of alleles, etc.), and/or any other number of times. All or portions of the method can be performed iteratively, concurrently, asynchronously, periodically, simultaneously, in series, in parallel, and/or at any other suitable time.

The method can be used with one or more cells. The cell (e.g., host cell) can be a human cell, any other animal cell, a bacteria cell, and/or any other cell. In examples, the cell can be a stem cell, a neuron, a retinal ganglion cell, an embryoid body, a fibroblast, a muscle cell, a kidney cell, and/or any other cell type. In a specific example, the cell can be an induced pluripotent stem cell (e.g., a human induced pluripotent stem cell). In an example, the cell can be a stem cell (e.g., an induced pluripotent stem cell) that undergoes one or more genetic modifications, wherein the cell can optionally be subsequently differentiated (e.g., into a neuron, an embryoid body, a fibroblast, a muscle cell, a kidney cell, etc.). In an illustrative example, the stem cell can be genetically modified, then differentiated into a neural precursor cell, and then matured into a neuron. The cell can be a genetically engineered cell (e.g., an edited cell) or a non-genetically engineered cell (e.g., unedited cell). In examples, the cell can be genetically engineered (e.g., engineered, genetically modified, generated, produced, edited, etc.) via all or a portion of the method (e.g., wherein the cell is a unedited cell prior to all or a portion of the method), genetically engineered prior to all or a portion of the method (e.g., a pre-edited cell), a combination thereof, and/or genetically engineered at any other time. In a specific example, a genetically engineered cell can be a cell that includes one or more genetically engineered sequences (e.g., a genetically engineered genome, one or more genetically engineered alleles, etc.). In a specific example, the cell can be a CIITA knockout cell. The cell can optionally be a part of a cell line, be used to produce a cell line (e.g., the cell is a parent cell for a cell line), and/or otherwise used. As used herein, the cell can refer to the original genetically modified cell and/or a cell derived from a cell line for the original genetically modified cell. The cell preferably includes one or more copies of a B2M gene (e.g., a native B2M gene), wherein all or portions of the method can be used to edit the B2M gene. However, the cell can alternatively not include a B2M gene (e.g., wherein the method introduces a B2M gene into the cell). The B2M gene is preferably located within a genome of the cell (e.g., at a B2M gene locus), but can be otherwise located. In an example, the cell includes two B2M alleles (e.g., located on different chromosomes).

The cell can be delivered to a subject (e.g., implantation, transplantation, engraftment, cell therapy administration, etc.), used for experimentation and/or other testing, and/or otherwise used. In a first variant, the cell can be used in a cell-based therapy (e.g., immunotherapy). In specific examples, the cell can be used as cell therapy (e.g., cell replacement) for: parkinsons, Amyotrophic Lateral Sclerosis (ALS), spinal cord injury, diabetes (e.g., cell replacement for pancreatic cells), epilepsy, and/or other injuries or diseases. In a second variant, the cell can be used in drug delivery factories (e.g., the cell can be used for drug delivery). In a third variant, the cell can be implanted in a tissue (e.g., brain, retina, etc.). For example, the cell can be seeded into an implant (e.g., neural implant, retinal implant, etc.). In a specific example, the implant can be a neural interface device (e.g., a brain-computer interface implant), wherein the cell can be used as an interface to neurons (e.g., directly interface with native neurons, indirectly interface with neurons via retinal ganglion cells, etc.). In a specific example, the neural interface device can be configured to be implanted on a surface of a brain. In specific examples, the one or more cells can be seeded into an implant as described in U.S. application Ser. No. 18/970,461 filed 5 Dec. 2024 and/or U.S. application Ser. No. 18/740,854 filed 12 Jun. 2024, each of which is incorporated in its entirety by this reference.

In an example, the cell can include a first nucleotide sequence comprising a B2M:HLA-E fusion gene (e.g., as described below) and a second nucleotide sequence encoding a channelrhodopsin (e.g., as described below). The B2M:HLA-E fusion gene can provide the cell with safeguarded hypoimmunogenicity, which can optionally enable the cell to be implanted. The channelrhodopsin can activate in response to receiving a light signal (e.g., from an external projector system, from an implant, etc.), which can optionally enable the cell to transmit information to native cells (e.g., neurons) at the implantation location. In an example, a neural interface device can include the one or more cells seeded into a set of scaffolds (e.g., wherein each cell is seeded between two adjacent scaffolds of the set of scaffolds). The one or more cells can optionally grow projections (e.g., axons, dendrites, etc.) out of the set of scaffolds to interface with the native neurons of the brain. In a specific example, the set of scaffolds can include: a recording electrode configured to receive an electrical signal from a cell; and a μLED configured to transmit a light signal to the cell, wherein the light signal activates the channelrhodopsin. In an illustrative example, each scaffold includes an array of recording electrodes (e.g., on one face of the scaffold) interfacing with a set of cells (e.g., a subset therein), and an array of μLEDs (e.g., on the opposing face of the scaffold) interfacing with the set of cells (e.g., a different subset therein).

As used herein, “sequence” preferably refers to a DNA sequence, but can additionally or alternatively refer to an RNA sequence and/or any other genomic sequence. As used herein, substantially contiguous (e.g., adjacent) sequences can be separated by less than a threshold number of base pairs, wherein the threshold can be between 1 bp-100 bp or any range or value therebetween (e.g., 1 bp, 2 bp, 5 bp, 10 bp, 50 bp, etc.). As used herein, substantially identical sequences can differ by less than a threshold number of bases in the respective sequences, wherein the threshold can be between 1 base-100 bases or any range or value therebetween (e.g., 1 base, 2 bases, 5 bases, 10 bases, etc.).

As used herein, a first sequence and a second sequence bounding (e.g., flanking) a third sequence can refer to an arrangement of sequences where the third sequence is located between the first sequence and the second sequence (e.g., within a vector, within a genome, etc.). In this arrangement, the first sequence and the second sequence are preferably each contiguous with (e.g., adjacent to) the third sequence, but alternatively can be noncontiguous with (e.g., nonadjacent to) the third sequence (e.g., one or more additional sequences can be located between the first sequence and the third sequence and/or between the third sequence and the second sequence).

4.1. Integrating a Selection Sequence into a Cell Genome S100.

The method can optionally include integrating a selection sequence into a cell genome S100, which functions to provide a pair of recombination sites (e.g., a landing site) that can subsequently be used to replace all or a portion of the selection sequence for an insertion sequence, wherein successful integration of the recombination site pair and/or replacement with the insertion sequence can be confirmed using cell selection methods. S100 can be performed before S200 and/or at any other time. S100 can be performed at a single locus (e.g., a single B2M gene locus corresponding to one B2M allele), at multiple loci (e.g., two B2M gene loci corresponding to two B2M alleles), and/or any other set of loci. Multiple instances of S100 (e.g., editing multiple B2M gene loci) can be performed iteratively, concurrently, asynchronously, periodically, simultaneously, in series (e.g., with a cell selection performed after each iteration of S100), in parallel, and/or otherwise performed.

The selection sequence can include an optional promoter, one or more selectable markers, a pair of recombination sites, and/or any other sequences.

The optional promoter can function to drive and/or increase the expression of the selectable marker(s) in the selection sequence. The optional promoter is preferably located on the 5′ side of the selectable marker(s) in the selection sequence, but can additionally or alternatively be located on the 3′ side of the selectable marker(s). The optional promoter is preferably contiguous with and/or substantially contiguous with the selectable marker(s), but can alternatively be non-contiguous with the selectable marker(s). Examples of promoters include: EF-1α, CAG, CMV, synapsin I, and/or any other promoter sequence. As used herein, “promoter” can optionally refer to the promoter sequence (e.g., the DNA sequence of the promoter).

The one or more selectable markers can function to impart features to the cell that can be used to verify integration and/or removal of all or a portion of the selection sequence. The selectable markers are preferably located on the 3′ side of the promoter, but can be otherwise located within the selection sequence. The number of selectable markers in the selection sequence is preferably between 1-5 or any range or value therebetween (e.g., 1, 2, 3, 4, 5), but can alternatively be greater than 5. Examples of selectable markers include: fluorophore genes (e.g., GFP, eGFP, RFP, BFP, mCherry, etc.), antibiotic genes (e.g., puromycin N-acetyl-transferase, aminoglycoside kinase, hygromycin B phosphotransferase, Sh ble, etc.), and/or any other selectable marker.

The pair of recombination sites can function to provide a landing site to replace the selectable markers and/or promoter with the insertion sequence (e.g., performing a cassette exchange). The recombination sites can interface with a recombinase (e.g., as described in S200). The pair of recombination sites (e.g., attachment sites) are preferably bounding all other elements of the selection sequence, but can alternatively be bounding a portion of the selection sequence. Examples of recombination sites can include: attB (e.g., attB1, attB2, attB3, attB4, attB5, attB6, etc.), attP (e.g., attB1, attB2, attB3, attB4, attB5, attB6, etc.), attR (e.g., attR1, attR2, attR3, attR4, attR5, attR6, etc.), attL (e.g., attL1, attL2, attL3, attL4, attL5, attL6, etc.), LoxP, FRT, and/or any other recombination element. As used herein, “recombination site” can optionally refer to the recombination site sequence (e.g., the DNA sequence of the recombination site).

The recombination site pair preferably includes non-compatible recombination sites (e.g., the recombination site pair includes two orthogonal recombination sites), but can alternatively include compatible recombination sites (e.g., attB1 and attP1, attB2 and attP2, etc.; attL1 and attR1, attL2 and attR2, etc.). Compatible (e.g., complementary) recombination sites can recombine with one another (e.g., via an SSR, via an SSR and an RDF, etc.) to form hybrid recombination sites; non-compatible (e.g., non-complementary, orthogonal, etc.) recombination sites cannot recombine with one another. In an illustrative example, compatible recombination sites attB1 and attP1 (pre-recombination state) can recombine to form compatible recombination sites attL1 and attR1 (post-recombination state), respectively. In an illustrative example, attB1 is compatible with attP1; attB1 is not compatible with any attB site (e.g., attB1, attB2, attB3, attB4, attB5, attB6, etc.), any attL site, any attR site, attP2, attP3, attP4, attP5, and attP6. The recombination site pair preferably includes only a single type of recombination site (e.g., only attB sites, only attP sites, only attL sites, only attR sites, etc.), but can alternatively include different types of recombination sites (e.g., one attB site and one attP site, one attB site and one attL site, etc.). Each recombination site in the pair preferably includes a unique recombination site sequence (e.g., a different sequence from the other recombination site), but can alternatively include the same sequence. In a first example, the recombination site pair can include any two different attB sequences selected from: attB1 (TT mutant), attB2 (AG mutant), attB3 (AC mutant), attB4 (TG mutant), attB5 (CC mutant), and attB6 (TC mutant). In a second example, the recombination site pair can include any two different attL sequences selected from: attL1 (TT mutant), attL2 (AG mutant), attL3 (AC mutant), attL4 (TG mutant), attL5 (CC mutant), and attL6 (TC mutant).

The recombination sites in the recombination site pair can be inverted relative to one another (e.g., the recombination site pair is an inverted recombination site pair), or can alternatively not be inverted. An inverted recombination site pair in a sequence (e.g., in a genome, vector, etc.) can be a pair of recombination sites with opposite orientations in the sequence. The orientation of a recombination site can be defined relative to the interface between the recombination site and a unidirectional SSR; the unidirectional SSR would interface with each recombination site in an inverted recombination site pair in opposite directions. Additionally or alternatively, the orientation of an recombination site in an recombination site pair can be defined by a shared sequence between the two recombination sites in the pair; when the shared sequences are oriented in opposite directions, the recombination site are inverted relative to one another.

However, the selection sequence can be otherwise configured.

The selection sequence is preferably integrated using CRISPR methods (e.g., homology-directed repair, non-homologous end-joining, prime editing, etc.), but can alternatively be integrated using other genetic engineering methods (e.g., any other knock-in methods). For example, integrating the selection sequence can include transfecting the cell with a vector carrying the selection sequence and optionally one or more additional sequences (e.g., guide sequences, Cas9, reverse transcriptase, etc.). In a specific example, the guide sequence can direct the selection sequence to integrate at a target within the B2M gene (e.g., target sequence and/or target location). In an illustrative example, the selection sequence is integrated using homology-directed repair (HDR) after double-stranded DNA cleavage.

In a first variant, integrating the selection sequence includes replacing a target sequence of the cell genome with the selection sequence. The target sequence preferably includes all or a portion of the B2M gene (e.g., the selection sequence can be integrated at the B2M gene locus). However, the target sequence can alternatively not include any of the B2M gene (e.g., the B2M gene can be knocked out, and the selection sequence can be integrated at another location in the cell genome). The target sequence can be a target sequence as described in S200 and/or any other target sequence. For example, the selection sequence can replace all or a portion of the B2M gene using methods described in S200.

In a second variant, integrating the selection sequence includes inserting the selection sequence into the cell genome at a target location. The target location is preferably a target location in the B2M gene (e.g., the selection sequence can be integrated at the B2M gene locus). However, the target location can alternatively not be within B2M gene (e.g., the B2M gene can be knocked out, and the selection sequence can be integrated at another location in the cell genome). The target location can be a target location as described in S200 and/or any other target sequence. For example, the selection sequence can be inserted into the B2M gene using methods described in S200.

The selection sequence can additionally or alternatively be integrated into the B2M gene as part of the insertion sequence (e.g., as described in S200).

However, the selection sequence can be otherwise integrated.

4.2. Integrating an Insertion Sequence into a Cell Genome S200.

Integrating an insertion sequence into the cell genome S200 functions to generate a genetically engineered sequence, which can provide a B2M:HLA fusion gene that can be translated into a B2M:HLA fusion protein (contributing to hypoimmunogenicity of the cell) and/or couple a B2M:HLA fusion gene to a kill switch gene. S200 can be performed after S100 and/or at any other time. S200 can be performed at a single locus (e.g., a single B2M gene locus corresponding to one B2M allele), at multiple loci (e.g., two B2M gene loci corresponding to two B2M alleles), and/or any other set of loci. Multiple instances of S200 (e.g., editing multiple B2M gene loci) can be performed iteratively, concurrently, asynchronously, periodically, simultaneously, in series (e.g., with a cell selection performed after each iteration of S200), in parallel, and/or otherwise performed. In first example, one B2M allele can be edited to include a first B2M:HLA fusion gene in a first instance of S200, and the other B2M allele can be edited to include a second B2M:HLA fusion gene in a second instance of S200. In a specific example, the two B2M:HLA fusion gene can be: two B2M:HLA-E fusion genes, two B2M:HLA-G fusion genes, one B2M:HLA-E fusion genes and one B2M:HLA-G fusion genes, and/or any other B2M:HLA fusion genes. In a specific example, a first insertion sequence can be integrated within a first B2M gene of the cell, and a second insertion sequence can be integrated within a second B2M gene of the cell, the second B2M gene located on a different chromosome than the B2M gene.

The insertion sequence can include: an HLA gene, a kill switch gene, and a multicistronic element. The insertion sequence can optionally include: one or more replacement B2M sequences (e.g., B2M sequences that are not native to the cell genome), a linker, a selection sequence, recombination sites, and/or any other DNA sequences.

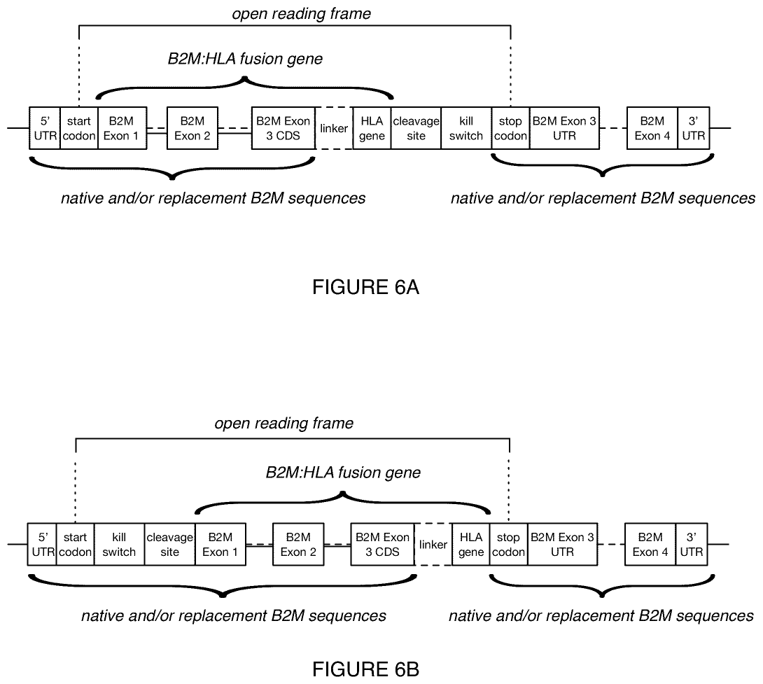

Integrating an insertion sequence can generate and/or modify a genetically engineered sequence (e.g., edited sequence) in the cell genome. Examples are shown in FIG. 6A and FIG. 6B. The genetically engineered sequence can include all or a portion of the insertion sequence (after integration of the insertion sequence). The genetically engineered sequence can include a B2M:HLA fusion gene, a kill switch gene, and a multicistronic element. The genetically engineered sequence can optionally include: one or more native and/or replacement B2M sequences, a linker, a selection sequence, recombination sites, and/or any other DNA sequences. The genetically engineered sequence can be located at the B2M locus and/or a different locus in the cell genome. The components of the insertion sequences are preferably simultaneously integrated into the cell genome (e.g., the insertion sequence is integrated as single, contiguous sequence). In a specific example, the genetically engineered sequence can be generated by simultaneously integrating an HLA gene, the multicistronic element, and the kill switch gene into a genome of the genetically engineered cell.

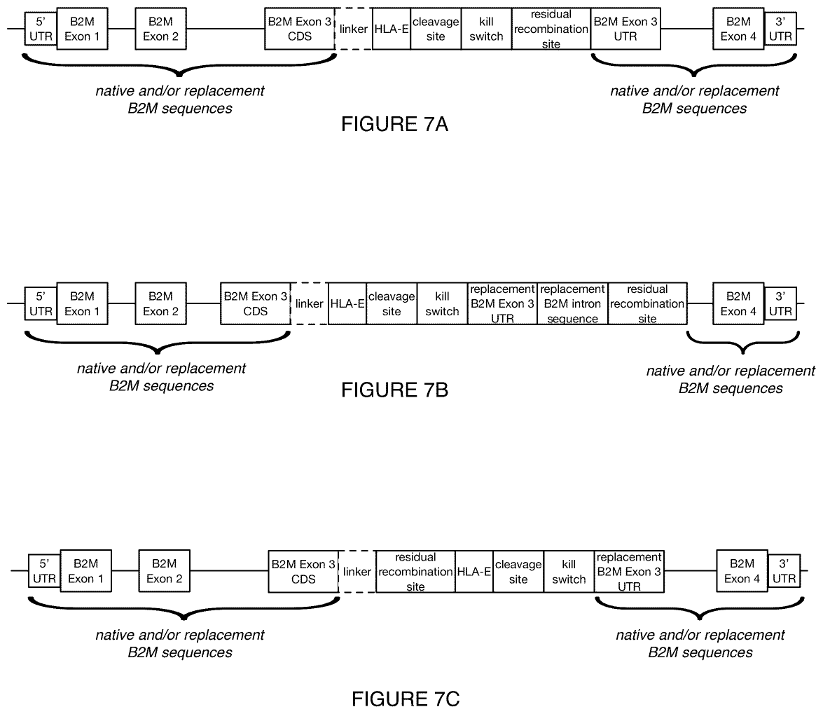

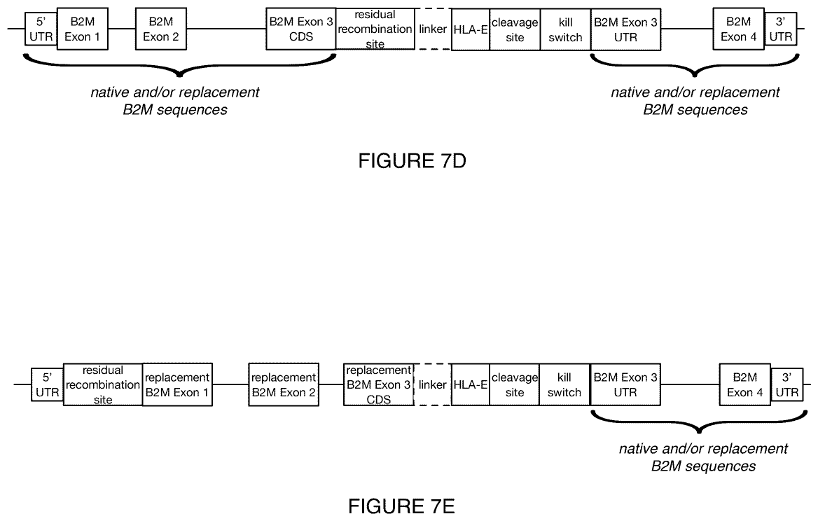

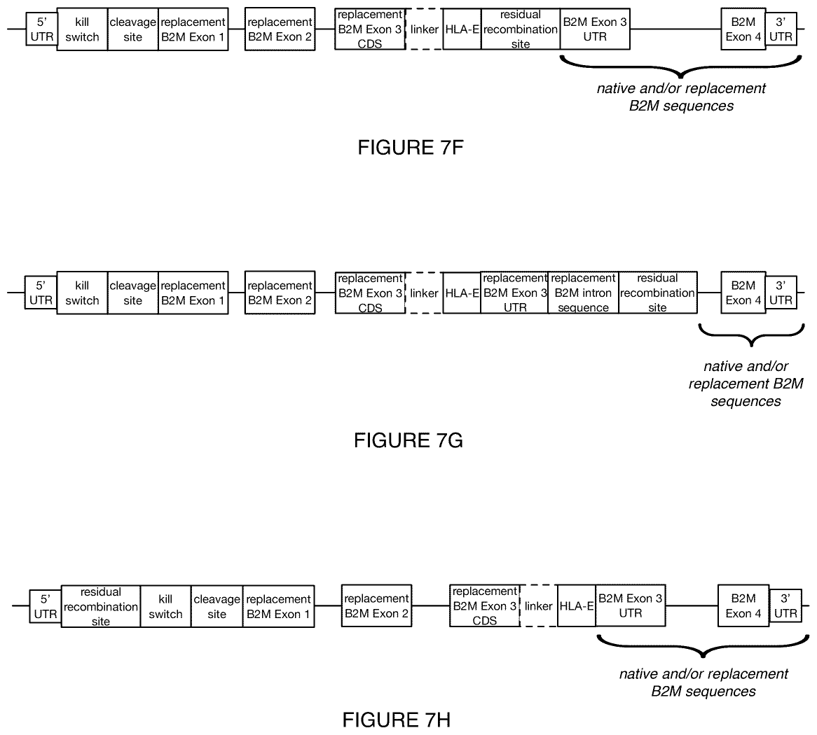

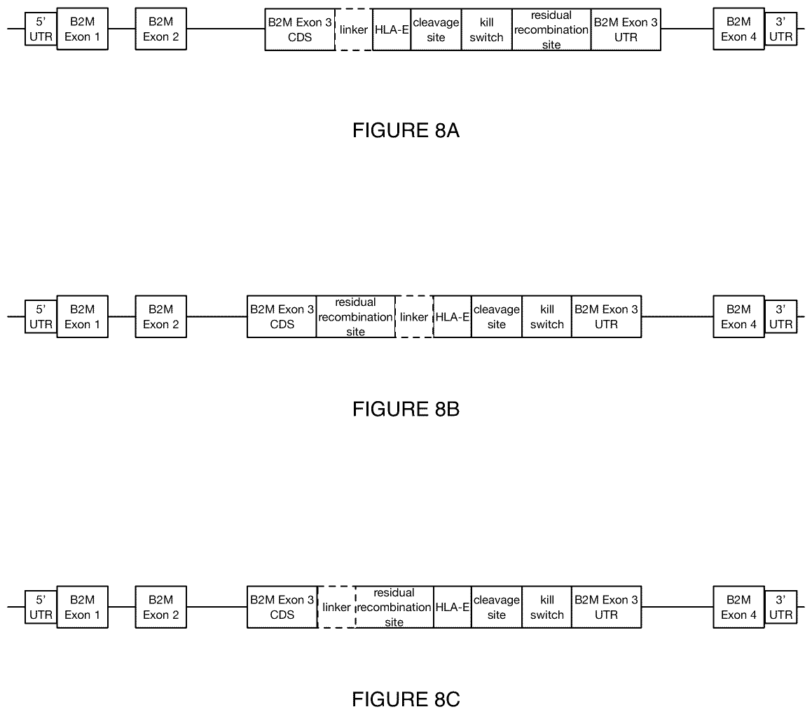

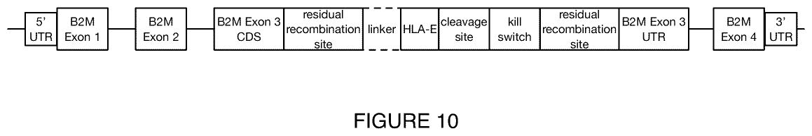

The optional linker (e.g., linker sequence), when translated into a linker peptide, functions to link the HLA protein to the B2M protein, forming the B2M:HLA fusion protein. In an example, the linker peptide can function to link the HLA protein to the C-terminus of the (native and/or replacement) B2M protein, wherein the resulting B2M:HLA fusion protein can include the B2M protein on the N-terminus side of the linker peptide (e.g., from the N-terminus of B2M full protein sequence through the C-terminus of B2M full protein sequence), the linker peptide, and the HLA protein. The linker peptide preferably covalently bonds the B2M protein to the HLA protein (e.g., via one or more peptide bonds), but can additionally or alternatively be include ionic bonds and/or any other bond. In an example, the linker peptide includes a non-cleavable sequence of amino acids (e.g., such that there are no cleavable sites within the B2M:HLA fusion protein). The linker peptide is preferably a single peptide, but can alternatively be a polypeptide. The linker can optionally be less than a threshold size; the threshold can be between 5 bp-1000 bp or any range or value therebetween (e.g., 10 bp, 20 bp, 50 bp, 100 bp, etc.), but can alternatively be less than 5 bp or greater than 1000 bp. Examples of linkers can include: (G4S)4, (G4S)3, (G3S)4, selectable markers, recombination sites, any other sequence. In a specific example, the linker can be or include a residual recombination site after selectable marker excision (e.g., examples in FIG. 7C, FIG. 7D, FIG. 8B, FIG. 8C, and FIG. 10). The linker can be located between the HLA gene and all or part of the B2M gene (e.g., native and/or replacement B2M sequences). In an example, the linker can be located between the 3′ terminus of the B2M exon 3 sequence and the 5′ terminus of the HLA gene (e.g., where the HLA gene is on the 3′ side of B2M exon 3). In a specific example, the linker can be on the 3′ side of the B2M exon 3 coding sequence and/or on the 5′ side of the B2M exon 3 stop codon. However, the linker can be otherwise positioned and/or configured.

The HLA gene functions in conjunction with the B2M gene to contribute to hypoimmunogenicity of the cell. The HLA gene is preferably a gene (e.g., a single gene, a complex of genes, etc.) for an HLA class I protein (e.g., corresponding to major histocompatibility complex (MHC) class I molecules). However, the HLA gene can alternatively be a gene for other HLA proteins. In specific examples, the HLA gene can be an HLA-E gene (corresponding to an HLA-E protein), an HLA-F gene (corresponding to an HLA-F protein), and/or an HLA-G gene (corresponding to an HLA-G protein). The HLA gene can be located on the 3′ side of a B2M exon 3 sequence, and optionally separated from the B2M exon 3 sequence by the linker (e.g., without any other intervening sequences). In a specific example, the HLA gene is located at the 3′ terminus of the linker. In a specific example, the HLA gene can be on the 3′ side of the B2M exon 3 coding sequence and/or on the 5′ side of the B2M exon 3 stop codon (e.g., between the 3′ terminus of the B2M exon 3 coding sequence and the B2M exon 3 stop codon). However, the HLA gene can be otherwise positioned and/or configured.

The kill switch gene functions to provide a means to induce cell death (e.g., if the cell become cancerous after editing). The kill switch gene (e.g., killswitch gene, suicide gene, etc.) can be a gene for a kill switch protein (e.g., killswitch protein, suicide protein, etc.) and/or a gene that marks the cell for killing (e.g., CD20, which is targeted by rituximab/mAb). Examples of kill switch genes and/or proteins (e.g., enzymes) thereof can include: Delta-Thymidine Kinase, iCasp9, RapaCasp, CD20, a drug-inducible kill switch protein, an activator for a kill switch protein, one or more proteins in a kill switch system, any other kill switch protein. In the genetically engineered sequence, the kill switch gene is preferably within the same open reading frame (ORF) as the B2M:HLA fusion gene and/or the multicistronic element such that the kill switch gene is co-transcribed with the B2M:HLA fusion gene and the multicistronic element (e.g., with no stop codons between B2M:HLA fusion gene, the multicistronic element, and the kill switch gene), but can alternatively be located in a different ORF. Examples are shown in FIG. 6A and FIG. 6B. In a specific example, the killswitch gene can be co-transcribed with the multicistronic element and the B2M:HLA fusion gene and/or sequences therein (e.g., the HLA gene). The kill switch gene can optionally include a start and/or stop codon. In a specific example, the kill switch gene includes a stop codon at the 3′ terminus of the kill switch gene when positioned on the 3′ side of the B2M:HLA fusion gene (e.g., to terminate the ORF encompassing the B2M:HLA fusion gene). The kill switch gene can be located on the 3′ side of the B2M:HLA fusion gene (e.g., on the 3′ side of HLA gene) or on the 5′ side of the B2M:HLA fusion gene (e.g., on the 5′ side of all or part of B2M exon 1). The kill switch gene is preferably contiguous with and/or substantially contiguous with the multicistronic element (e.g., in the insertion sequence and/or in the genetically engineered sequence), but can be non-contiguous to the multicistronic element. In an example, the multicistronic element can be positioned between the kill switch gene and the B2M:HLA fusion gene (e.g., in the genetically engineered sequence). In a first specific example, the kill switch gene can be located on the 3′ side of the multicistronic element when the multicistronic element is positioned on the 3′ side of the B2M:HLA fusion gene. In a second example, the kill switch gene can be located on the 5′ side of the multicistronic element when the multicistronic element is positioned on the 5′ side of the B2M:HLA fusion gene. In an example, the multicistronic element can be positioned between the kill switch gene and the HLA gene (e.g., in the insertion sequence). However, the kill switch gene can be otherwise positioned and/or configured.

The multicistronic element (e.g., cleavage site) functions to generate separate B2M:HLA fusion and kill switch proteins, such that the kill switch protein remains functional inside the cell and does not travel to the cell surface with the B2M:HLA fusion protein. As used herein, a “multicistronic element” can refer to a DNA sequence, an RNA element generated therefrom, and/or a peptide generated therefrom. The multicistronic element can generate separate proteins using: co-translational separation of the B2M:HLA fusion protein from the kill switch protein (e.g., 2A “self-cleaving” peptides), ribosomal skipping between the B2M:HLA fusion sequence and the kill switch sequence during translation (e.g., internal ribosomal entry site, or IRES), cleaving the B2M:HLA fusion protein from the kill switch protein via an enzyme (e.g., TEV protease cleavage site), and/or any other cleavage mechanism. Examples of multicistronic elements include: internal ribosome entry site (IRES), T2A, P2A, E2A, F2A, other cleavage sites, and/or any other cleavage sequence. In the insertion sequence, the HLA gene, the multicistronic element, and the kill switch gene preferably form a contiguous sequence, but can alternatively form a non-contiguous sequence. In a specific example, the multicistronic element is contiguous with the HLA gene and is contiguous with killswitch gene. The multicistronic element is preferably located between the HLA gene and the kill switch gene, wherein the HLA gene can be on the 5′ or the 3′ side of the multicistronic element with the kill switch gene on the opposing side of the multicistronic element. In a specific example, the insertion sequence can include: an HLA gene, a multicistronic element, and a killswitch gene, wherein the multicistronic element is located between the killswitch gene and the HLA class I gene in the insertion sequence. In the genetically engineered sequence, the B2M:HLA fusion gene, the multicistronic element, and the kill switch gene preferably form a contiguous sequence, but can alternatively form a non-contiguous sequence. The multicistronic element is preferably located between the B2M:HLA fusion gene and the kill switch gene, wherein the B2M:HLA fusion gene can be on the 5′ or the 3′ side of the multicistronic element with the kill switch gene on the opposing side of the multicistronic element. However, the multicistronic element can be otherwise positioned and/or configured.

The optional replacement B2M sequence(s) can function to replace all or part of the B2M gene (e.g., at the same location and/or at a different location in the cell genome). The replacement B2M sequence (e.g., non-native B2M sequence) is preferably identical to a corresponding segment of the native B2M gene (e.g., a replacement B2M exon 3 sequence is identical to the native B2M exon 3), but can alternatively be different from the corresponding segment of the native B2M gene (e.g., a replacement B2M sequence including B2M exon 1, B2M exon 2, and B2M exon 3, without B2M introns). In an example, the replacement B2M sequence includes all or part of exon 3 of the B2M gene. In a specific example, a first replacement B2M sequence includes all or part of the B2M exon 3 coding sequence (CDS), and a second replacement B2M sequence includes all or part of the B2M exon 3 untranslated region (UTR) and/or all or part of the B2M intron 3. In a second specific example, the replacement B2M sequence includes all or part of exon 1 (e.g., not including the 5′ UTR), exon 2, and exon 3 (e.g., only the exon 3 CDS) of the B2M gene. One or more replacement B2M gene sequences can optionally be located (sequentially or non-sequentially) within the insertion sequence. For example, the insertion sequence can include a replacement B2M sequence, wherein integrating the insertion sequence into the B2M gene includes replacing a native B2M sequence in the B2M gene with the replacement B2M sequence (e.g., wherein the replacement B2M sequence and the native B2M sequence are substantially identical). In a first example, the replacement B2M sequence can be located on the 5′ side of the linker and/or the HLA gene (e.g., an exon 3 CDS replacement sequence is positioned on the 5′ side of the linker and the HLA gene). In a second example, the replacement B2M sequence can be located on the 3′ side of the kill switch gene. In a first specific example, a B2M exon 3 UTR replacement and/or a partial B2M intron 3 replacement is positioned on the 3′ side of the kill switch gene and multicistronic element (e.g., wherein the multicistronic element and kill switch gene are positioned on the 3′ side of the B2M:HLA fusion gene sequence). In a second specific example, a B2M replacement sequence including B2M exon 1, exon 2, and exon 3 (e.g., and optionally including intron 1 and/or intron 2) is positioned on the 3′ side of the kill switch gene and multicistronic element. However, the replacement B2M sequence(s) can be otherwise positioned and/or configured.

The optional selection sequence can function to confirm the insertion sequence is integrated into the B2M gene. In examples, the selection sequence can be located: on the 3′ side of the kill switch gene (e.g., after a stop codon in the kill switch gene); on the 3′ side of a native and/or replacement B2M sequence (e.g., a B2M exon 3 CDS, a B2M exon 3 UTR, a B2M intron 3 sequence, etc.); on the 5′ side of a native and/or replacement B2M sequence (e.g., a B2M exon 1 sequence, a B2M exon 3 UTR, a B2M intron 3 sequence, etc.); within or adjacent to the linker (on either the 3′ side or the 5′ side of the linker); and/or otherwise located. The selection sequence is preferably located outside of the genetically engineered sequence that includes the kill switch gene, the multicistronic element, and the B2M:HLA fusion gene, but can alternatively be located within the genetically engineered sequence (e.g., wherein a residual recombination site after excision of selectable markers in the selection sequence functions as all or part of the linker) or can be otherwise located. The selection sequence can be located in the same ORF of the kill switch gene, multicistronic element, and B2M:HLA fusion gene, or can be located in a different ORF (e.g., a selection sequence located on the 3′ side of the kill switch gene after a stop codon; a selection sequence in a separate ORF that breaks the kill switch gene, multicistronic element, and B2M:HLA fusion gene ORF; etc.). The selection sequence can include an optional promoter, one or more selectable markers, and a pair of recombination sites. For example, the selection sequence can include a selection sequence as described in S100 and/or components therein. The pair of recombination sites (e.g., bounding the selectable markers and/or promoter) can function to enable excision of the selectable markers (e.g., via S400). In a specific example, the insertion sequence includes a selection sequence including a pair of recombination sites bounding a selectable marker (e.g., wherein the method includes excising the selectable marker using a recombinase). The pair of recombination sites are preferably compatible (e.g., can combine to form a residual recombination site), but can alternatively not be compatible. For example, the pair of recombination sites can include one attB site and one attP site. However, the selection sequence can be otherwise positioned and/or configured.

The B2M:HLA fusion gene functions to contribute to hypoimmunogenicity of the cell. The B2M:HLA fusion gene preferably includes a B2M sequence (e.g., all or a portion of the B2M gene) and the HLA gene. The B2M sequence can include native B2M sequences and/or replacement B2M sequences (e.g., non-native B2M sequences from the insertion sequence). The B2M sequence and the HLA gene can be located in the same ORF such that the HLA gene will be co-transcribed with the B2M sequence, and the resultant HLA protein will be coupled to the B2M protein. The B2M:HLA fusion gene can optionally include a linker between the B2M sequence and the HLA gene. In a specific example, the B2M sequence in the B2M:HLA fusion gene can include B2M exon 1, B2M, exon 2, and B2M exon 3. The B2M sequence in the B2M:HLA fusion gene can be identical to a segment of a native, unmodified B2M gene sequence (e.g., the B2M:HLA fusion gene includes B2M introns between exon 1, exon 2, and exon 3) or different (e.g., all or a portion of the B2M introns between exon 1, exon 2, and exon 3 can be missing or modified). In a first variant, the B2M:HLA fusion gene can be generated via integration of the insertion sequence. For example, the insertion sequence can be integrated into the (native) B2M gene of a cell genome, wherein all or a portion of the B2M sequence in the B2M:HLA fusion gene can be native sequences. In a second variant, the insertion sequence can include a B2M:HLA fusion gene (e.g., a pre-generated B2M:HLA fusion gene), wherein the B2M:HLA fusion gene is integrated into the cell genome. For example, the B2M sequence in the B2M:HLA fusion gene can be a replacement sequence. However, the B2M:HLA fusion gene can be otherwise positioned and/or configured.

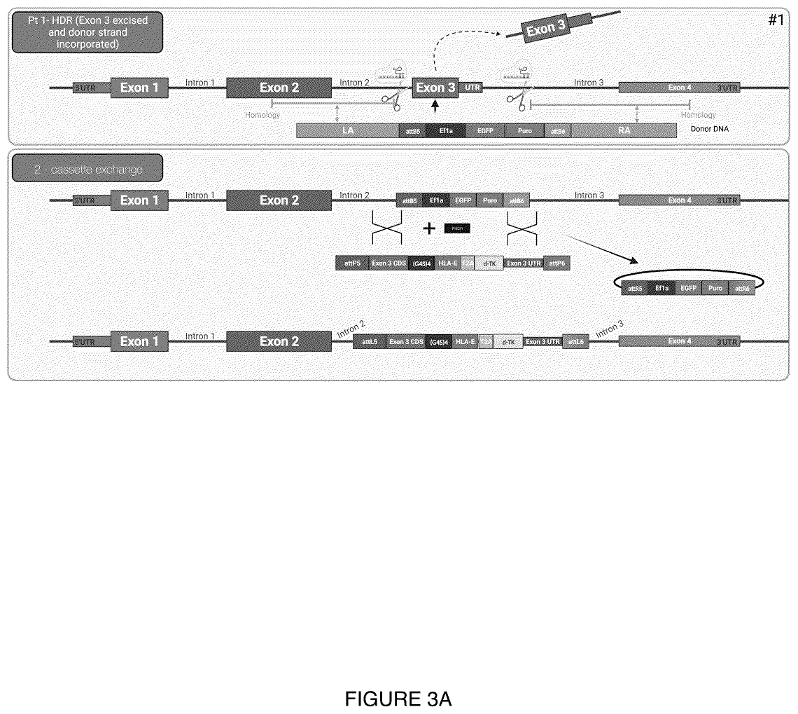

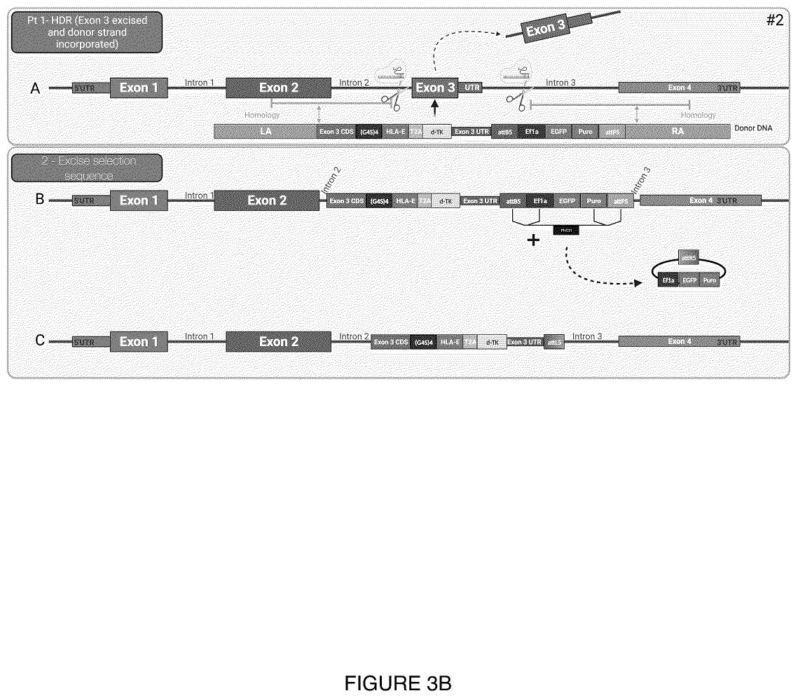

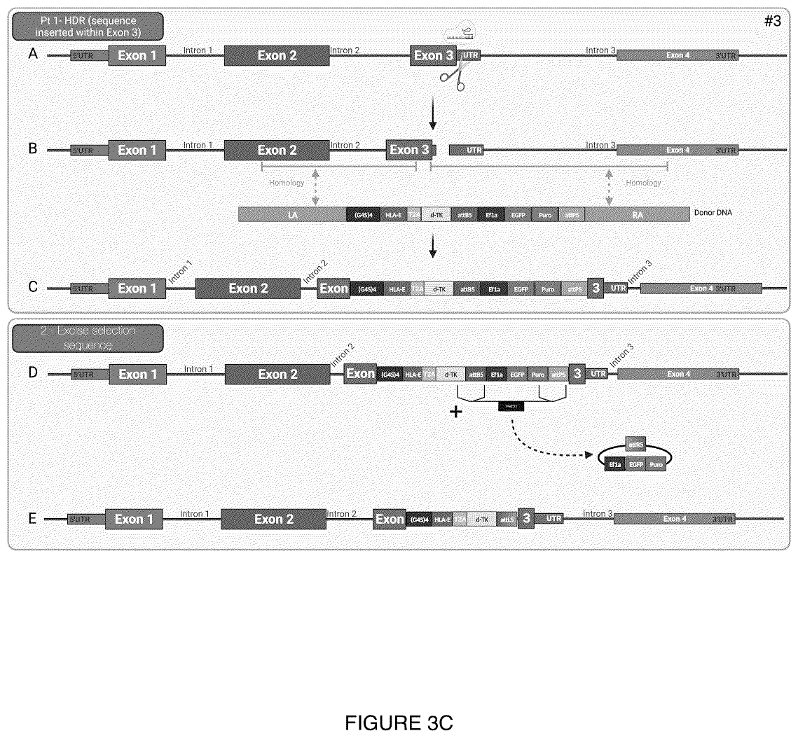

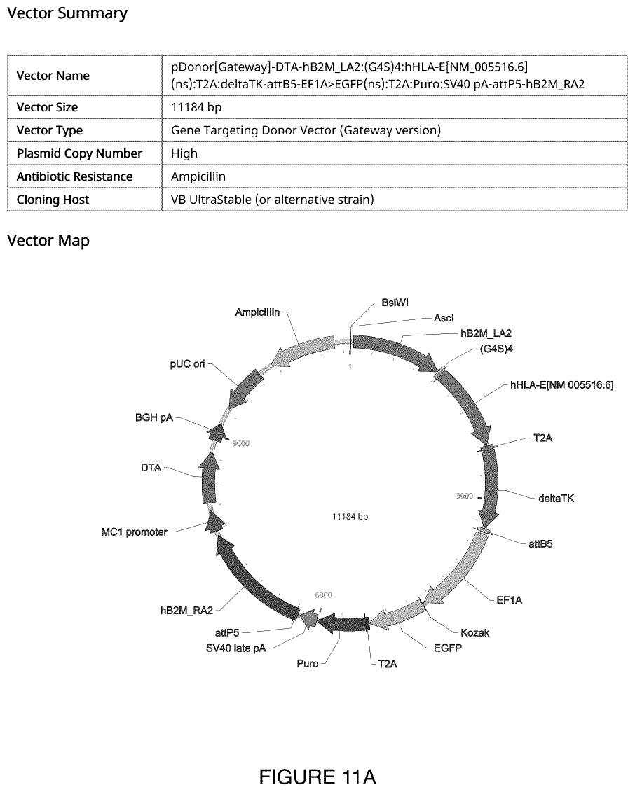

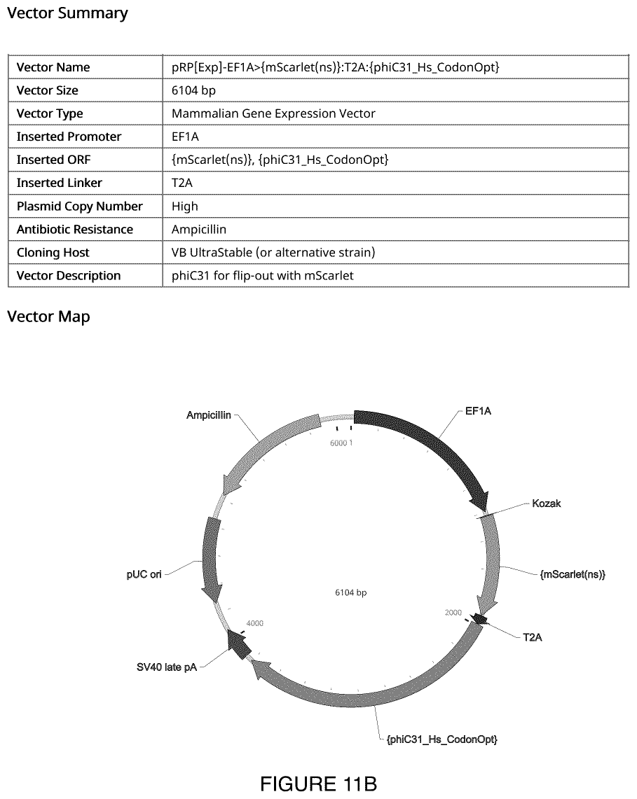

Integrating the insertion sequence into the B2M gene can optionally include transfecting the cell using a vector (e.g., plasmid, construct, etc.) carrying the insertion sequence and optionally one or more additional sequences (e.g., guide sequences, Cas9, reverse transcriptase, recombination sites bounding the insertion sequence, etc.). An example of the vector is shown in FIG. 11A. The insertion sequence can be integrated into the B2M gene using CRISPR methods (e.g., any method in S100), a recombinase, and/or any other genetic engineering methods. Examples are shown in FIG. 3A, FIG. 3B, and FIG. 3C. The recombinase is preferably a site-specific recombinase (SSR) (e.g., serine integrase, tyrosine integrase, etc.), but can alternatively be any other recombinase (e.g., any other integrase) and/or any other enzyme. The recombinase is preferably a unidirectional recombinase, but can alternatively not be unidirectional. Examples of recombinases include: phiC31, Bxb1, Peaches, Theia, Benedict, Veracruz, Rebeuca, Cre, FLP, and/or any other recombinase.

In a first variant, integrating the insertion sequence into the cell genome includes replacing a target sequence of the cell genome with the insertion sequence. All or a portion of the target sequence can optionally be excised. The target sequence preferably includes all or a portion of the B2M gene (e.g., the insertion sequence can be integrated at the B2M gene locus). For example, the genetically engineered sequence can be an edited B2M gene with the insertion sequence replacing all or part of the B2M gene. However, the target sequence can alternatively not include any of the B2M gene (e.g., the B2M gene can be knocked out, and the insertion sequence can be integrated at another location in the cell genome). The target sequence preferably partially or fully overlaps with an open reading frame (ORF) of the B2M gene, but can alternatively not overlap with an ORF of the B2M gene. For example, the target sequence can include all or a portion of the ORF and optionally additional sequence(s) beyond the ORF (e.g., sequence(s) on either side of the ORF). An open reading frame of the B2M gene can include the sequence between the B2M exon 1 start codon and the B2M exon 3 stop codon. In examples, the target sequence can include all or a portion of: exon 1 of the B2M gene (B2M exon 1), exon 2 of the B2M gene (B2M exon 2), exon 3 of the B2M gene (B2M exon 3), one or more introns, and/or any other region of the B2M gene. In a first specific example, the target sequence includes a B2M sequence between a first locus in intron 2 and a second locus in intron 3. In a second specific example, the target sequence includes a B2M sequence between a first locus at or near the 5′ terminus of exon 1 (e.g., before the 5′ start of the 5′ UTR, within the 5′ UTR, at the 5′ terminus of exon 1 CDS, before the exon 1 start codon, after the exon 1 start codon, etc.) and a second locus at or near the 3′ terminus of exon 3 (e.g., at the 3′ terminus of the exon 3 CDS, within the exon 3 UTR, within intron 3, etc.). In an illustrative example, after integration, the insertion sequence is located on a 5′ side of a B2M exon 3 stop codon. Examples of a genetically engineered sequence (after selectable marker removal) where the insertion sequence replaced a target sequence of the cell genome are shown in FIG. 7A-7H.

In a second variant, integrating the insertion sequence into the cell genome includes inserting the insertion sequence into the cell genome at a target location. The target location is preferably a target location in the B2M gene (e.g., the selection sequence can be integrated at the B2M gene locus). For example, the genetically engineered sequence can be an edited B2M gene with the insertion sequence inserted into the B2M gene. However, the target location can alternatively not be within B2M gene (e.g., the B2M gene can be knocked out, and the selection sequence can be integrated at another location in the cell genome). The target location is preferably within an open reading frame (ORF) of the B2M gene, but can alternatively not be integrated into an ORF of the B2M gene (e.g., within the 5′ untranslated region, the within the 3′ untranslated region, within a different ORF, etc.). The target location is preferably within a coding sequence of the B2M gene, but can alternatively be within a noncoding region. In an example, the target location can be within a B2M exon (e.g., within the coding sequence of a B2M exon). In examples, the target location can be within B2M exon 1, B2M exon 2, or B2M exon 3. In a first example, the target location can be at or near the 3′ terminus of the B2M exon 3 sequence (e.g., immediately before the exon 3 UTR, on the 5′ side of the B2M exon 3 stop codon). In a specific example, the target location can be on the 3′ side of the B2M exon 3 coding sequence and/or on the 5′ side of the B2M exon 3 stop codon (e.g., between the 3′ terminus of the B2M exon 3 coding sequence and the B2M exon 3 stop codon; optionally contiguous with the exon 3 stop codon). In an illustrative example, after integration, the insertion sequence is located on a 5′ side of a B2M exon 3 stop codon. In a second example, the target location can be at or near the 5′ terminus of the B2M exon 1 sequence. In a specific example, the target location can be on the 5′ side of the B2m exon 1 coding sequence and/or on the 3′ side of the B2M exon 1 start codon (e.g., between the B2M exon 1 start codon and the 5′ terminus of the B2M exon 1 coding sequence; optionally contiguous with the exon 1 start codon). Examples of a genetically engineered sequence (after selectable marker removal) where the insertion sequence was inserted at a target location of the cell genome are shown in FIG. 8A, FIG. 8B, and FIG. 8C.

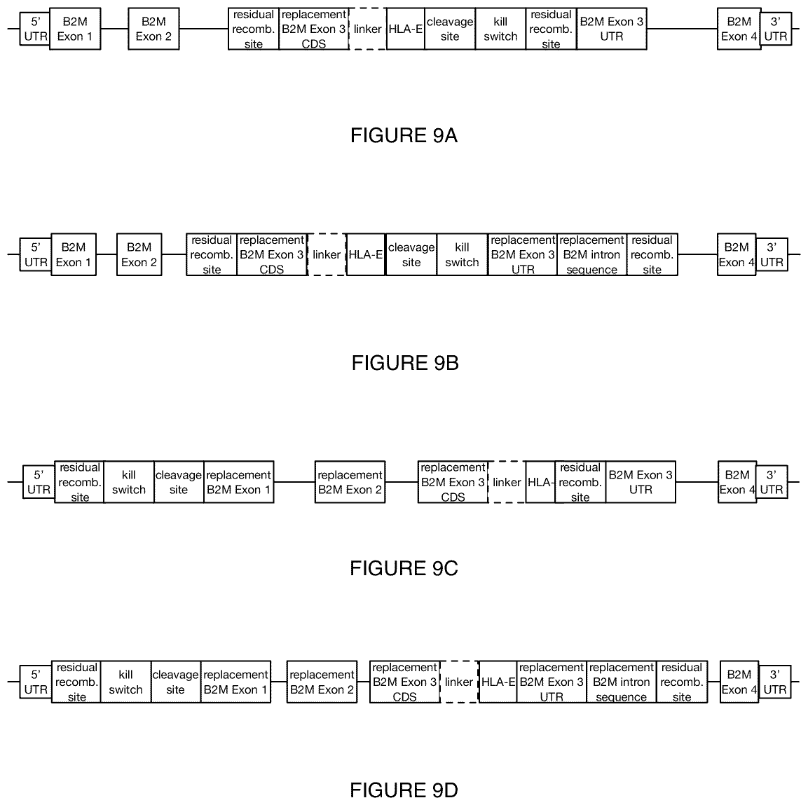

In a third variant, integrating the insertion sequence into the cell genome includes replacing the selection sequence (integrated into the cell genome in S100) with the insertion sequence. An example is shown in FIG. 3A. For example, using a recombinase, the selectable markers (and promoter) bounded by a first pair of recombination sites in the (edited) cell genome (e.g., in the edited B2M gene) can be exchanged with the insertion sequence bounded by a second pair recombination sites. The second pair of recombination sites is preferably compatible with (e.g., maps to) the first pair of recombination sites. For example, a first recombination site bounding the insertion sequence is compatible with a first recombination site in the selection sequence, and a second recombination site bounding the insertion sequence is compatible with a second recombination site in the selection sequence. The compatibility (e.g., mapping) is preferably unique (e.g., the first recombination site bounding the insertion sequence is not compatible with the second recombination site in the selection sequence), but can alternatively not be unique. In an illustrative example, selectable markers and a promoter are bounded by attB5 and attB6 in the selection sequence in the cell genome, and the insertion sequence is bounded by attP5 and attP6 in a transfection vector. Once exchanged, the insertion sequence is bounded by residual recombination sites attL5 and attL6 within the cell genome, and the selectable markers and promoter are bounded by attR5 and attR6 within a residual vector (e.g., non-integrated recombined vector). Examples of a genetically engineered sequence where the insertion sequence replaced the selection sequence are shown in FIG. 9A, FIG. 9B, FIG. 9C, FIG. 9D, and FIG. 10.

However, the insertion sequence can be otherwise integrated.

### 4.3. Performing a Cell Selection S300.

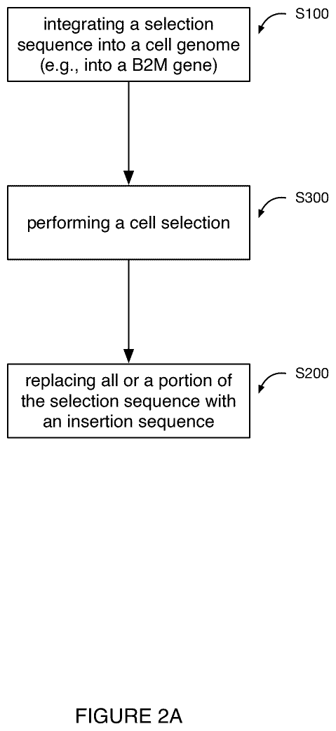

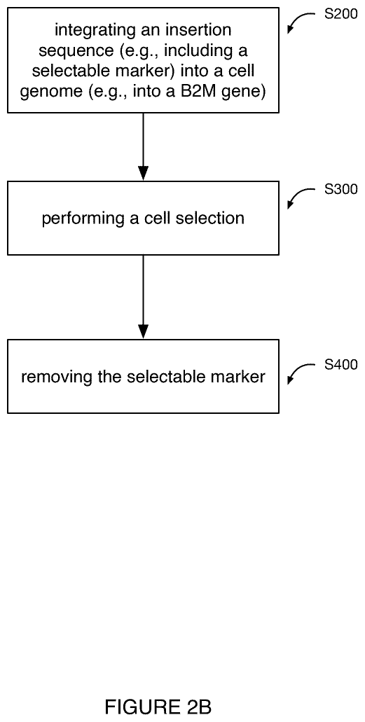

Performing a cell selection S300 functions to select one or more cells with a sequence of interest (e.g., the selection sequence, the insertion sequence, etc.) properly integrated into the cell genome (e.g., into the B2M gene). S300 can be performed after S100, after S200, after S400, and/or at any other time. Examples are shown in FIG. 2A and FIG. 2B.

In a first variant, performing the cell selection can include performing a positive selection. For example, performing the cell selection can include identifying one or more cells from a set of cells that include one or more selectable markers (e.g., identifying cells that fluoresce) and/or exhibit desired functionality (e.g., does not stimulate an immune response). In a second variant, performing the cell selection can include performing a negative selection. For example, performing the cell selection can include identifying one or more cells from a set of cells that does not include one or more selectable markers and/or does not exhibit desired functionality.

In a first specific example, the method includes: attempting an integration of an insertion sequence including a kill switch gene, a multicistronic element, an HLA gene, and a selection sequence into the B2M gene of a set of cells (e.g., S200), performing a positive selection to identify a subset of cells (within the set of cells) with the insertion sequence properly integrated, optionally attempting a removal of the selection sequence from the genomes of the subset of cells (e.g., S400), and optionally performing a negative selection to identify a second subset of cells (within the subset of cells) with the selection sequenced properly excised. In a second specific example, the method includes: attempting an integration of a selection sequence into the B2M gene of a set of cells (e.g., S100), performing a positive selection to identify a first subset of cells (within the set of cells) with the selection sequence properly integrated, attempting to replace the selectable markers in the genomes of the first subset of cells with an insertion sequence including a kill switch gene, a multicistronic element, and an HLA gene (e.g., S200), and optionally performing a negative selection to identify a second subset of cells (within the first subset of cells) with the insertion sequence properly integrated.

However, cell selection can be otherwise performed.

### 4.4. Removing a Selectable Marker S400.

The method can optionally include removing a selectable marker S400, which functions to eliminate one or more features imparted to the cell via selectable marker(s) (e.g., puromycin resistance, fluorescence, etc.). S400 can be performed after S100, after S200, during S200, after S300, and/or at any other time.

One or more selectable markers can be removed. The one or more selectable markers preferably includes all selectable markers integrated via S100 and/or S200 (e.g., all selectable markers in the genetically engineered sequence generated via S100 and/or S200). Alternatively, the one or more selectable markers can include a portion of the selectable markers integrated via S100 and/or S200 (e.g., a portion of the selectable markers in the genetically engineered sequence generated via S100 and/or S200). However, any other selectable markers can be removed.

The selectable marker(s) are preferably removed from a selection sequence (e.g., a selection sequence integrated in S100, a selection sequence within the insertion sequence integrated in S200, etc.), but can alternatively be removed from any other part of the cell genome. The selectable marker(s) are preferably removed using a recombinase corresponding to recombination sites in the selection sequence, but can alternatively be removed using any other genetic engineering method. For example, the selectable marker(s) can be removed using an SSR (e.g., a serine integrase, a tyrosine integrase, etc.). The recombinase is preferably a unidirectional recombinase, but can alternatively not be a unidirectional recombinase. In an illustrative example, the selectable marker(s) can be removed using phiC31.

In a first variant, removing the selectable marker(s) includes excising all or a portion of the selection sequence. For example, removing the selectable markers can include performing an excision between a pair of recombination sites in the selection sequence using a recombinase, wherein the two recombination sites recombine to form a residual recombination site (e.g., another recombination site, a hybrid recombination site, a post-recombination site, etc.). The recombinase can optionally be introduced using a vector; an example is shown in FIG. 11B. In a first specific example, an attB and attP recombination site pair bounding selectable markers in the B2M gene locus (e.g., in the genetically engineered sequence) can be transformed to a (single) attL recombination site, while the excised selectable markers form a residual vector (e.g., non-integrated recombined vector) with an attR recombination site. In a second specific example, a pair of two LoxP recombination sites bounding selectable markers in the B2M gene can be transformed to a single LoxP recombination site, while the excised selectable markers form a residual vector with a LoxP recombination site. In a second variant, removing the selectable marker(s) includes replacing all or part of the selection sequence (e.g., as described in S200).

However, the selection sequence can be otherwise removed.

### 4.5. Editing an Additional Gene S500.

The method can optionally include editing an additional gene S500, which functions to add or remove one or more target functionalities to the cell (e.g., immune-response functionalities, cell signaling functionalities, etc.). S500 can be performed before all or parts of the method (e.g., using a pre-edited cell in S100 and/or S200), after all/parts of the method (e.g., editing the hypoimmunogenic cell line after S300 and/or S400), and/or at any other time. S500 can be performed one or more times for one or more additional gene edits.

In a first variant, one or more genes of interest can be integrated (e.g., using any knock-in method) in the cell genome. For example, the gene(s) of interest can be integrated at a safe harbor site and/or any other locus. The integration locus is preferably not within the B2M gene, but can alternatively be within the B2M gene. In specific examples, genes of interest that can be integrated can include: opsins, transcription factors, selectable markers, kill switch genes, transcription factor, opsin, cell differentiation genes, cell adhesion molecule genes, hypoimmunogenicity genes, fluorophore genes, cell surface molecule expression genes, recombination sites, a landing site, any transgene, multiple genes (in sequence), and/or any other gene of interest. In a second variant, additional genes in the cell genome can be edited to remove immune-responsive signals and/or to increase immune-evading signals. Examples of genes that can be edited include: CIITA, NKG2 genes, other HLA genes or genes associated with HLA function, CR1, CD46, CD55, CD59, CD47, CD74, CD64, TAP1, B2M, and/or any other gene. In a first example, CIITA can be knocked out to eliminate or reduce expression of HLA Class II proteins. In a second example, the B2M gene can be knocked out in one or both B2M alleles in the cell to reduce or eliminate free-floating B2M proteins. In a first specific example, the B2M gene in one allele can be knocked out (e.g., homologous recombination, CRISPR-Cas9, TALENs, and/or any knock out method) while the B2M gene in the other allele is edited using all or parts of the method. In a second specific example, the B2M gene in both alleles can be knocked out. In this example, the insertion sequence can include all or a portion of a B2M gene (e.g., within a B2M:HLA fusion gene) such that the B2M:HLA fusion protein can be produced without the (native) B2M genes of the cell. In a third variant, S500 can include introducing a chromatin opening site (e.g., a sequence of DNA that promotes histone modifications that result in open chromatin) upstream of the B2M gene locus (e.g., on the 5′ side of the B2M:HLA fusion gene). In an example, this edit can maintain activity of the genetically engineered sequence (e.g., combatting epigenetic repression).

However, additional genes in the cell can be otherwise edited.

### 5. A Genetically Engineered Sequence

A sequence (e.g., a genome of a cell) can be optionally genetically engineered (e.g., engineered, edited, genetically modified, produced, generated, etc.) using all or parts of the method. For example, the sequence can be genetically engineered via one or more instances of S100, S200, S300, S400, and/or S500. In examples, the sequence can include a nucleic acid sequence (e.g., mRNA sequence, DNA sequence, etc.) that includes the respective genes (e.g., cistrons) described below. In examples, the sequence can increase the expression of protein complexes encoded by the respective genes described below relative to a parent cell, and optionally decrease the expression of some or all other protein complexes encoded by other genes (e.g., not described below) relative to the parent cell.

In variants, a genetically engineered sequence (e.g., genetically altered sequence) can include: a B2M:HLA fusion gene; a multicistronic element; and a kill switch gene. In an example, a genetically engineered sequence can include: a B2M:HLA fusion gene; a multicistronic element contiguous with the B2M:HLA fusion gene; and a kill switch gene contiguous with the multicistronic element. The multicistronic element is preferably located between the kill switch gene and the B2M:HLA fusion gene, but can be otherwise located. In a first specific example, the kill switch gene can be located on the 3′ side of the multicistronic element (e.g., contiguous with the 3′ terminus of the multicistronic element) and the B2M:HLA fusion gene can be located on the 5′ side of the multicistronic element (e.g., contiguous with the 5′ terminus of the multicistronic element); example shown in FIG. 4A. In a second specific example, the kill switch gene can be located on the 5′ side of the multicistronic element (e.g., contiguous with the 5′ terminus of the multicistronic element) and the B2M:HLA fusion gene can be located on the 3′ side of the multicistronic element (e.g., contiguous with the 3′ terminus of the multicistronic element); example shown in FIG. 4B. A native B2M gene of a cell can optionally be edited to be the genetically engineered sequence. The genetically engineered sequence can optionally include one or more non-native B2M sequences (e.g., replacement B2M sequences) replacing one or more B2M sequences native to the cell. In a specific example, the genetically engineered sequence can include a non-native B2M sequence replacing all or a portion of the native B2M gene of the cell.

In an example, the kill switch gene and the B2M-HLA fusion gene are each located in a shared open reading frame (e.g., the kill switch gene and the B2M-HLA fusion gene are co-transcribed, co-translated, etc.). In a specific example, the genetically engineered sequence generates a B2M:HLA fusion protein and a kill switch protein, separate from the B2M:HLA fusion protein (e.g., separate proteins can be generated due to the multicistronic element positioned between the B2M:HLA fusion protein and the kill switch protein). In a first example, the multicistronic element and kill switch gene are located within a B2M exon 3 sequence of the B2M-HLA fusion gene. In a specific example, the multicistronic element and kill switch gene can be on the 3′ side of the B2M exon 3 coding sequence and/or on the 5′ side of the B2M exon 3 stop codon (e.g., between the 3′ terminus of the B2M exon 3 coding sequence and the B2M exon 3 stop codon; optionally contiguous with the exon 3 stop codon). In a second example, the multicistronic element and kill switch gene are located within a B2M exon 1 sequence of the B2M-HLA fusion gene. In a specific example, the multicistronic element and kill switch gene can be on the 5′ side of the B2M exon 1 coding sequence and/or on the 3′ side of the B2M exon 1 start codon (e.g., between the B2M exon 1 start codon and the 5′ terminus of the B2M exon 1 coding sequence; contiguous with the exon 1 start codon).

The B2M:HLA fusion gene can include a B2M:HLA fusion gene, a B2M:HLA-G fusion gene, and/or any other B2M:HLA fusion gene. In a specific example, the B2M:HLA fusion gene can include all or a portion of a B2M gene (e.g., including native and/or nonnative B2M exon 1, B2M exon 2, and B2M exon 3 sequences) fused to all or a portion of an HLA class I gene (e.g., fused via a linker).

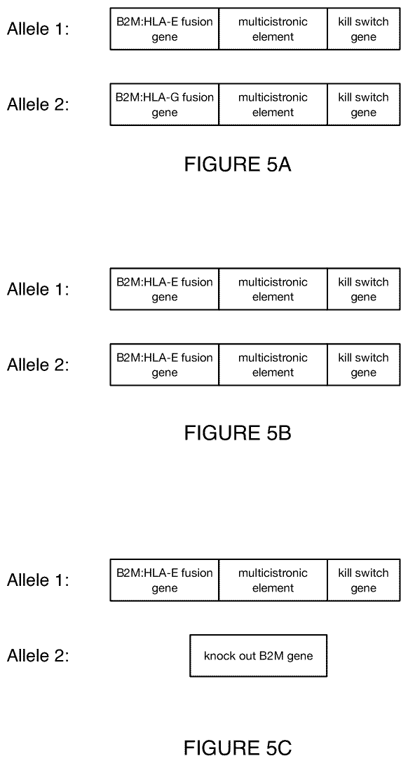

In variants, a cell can include two genetically engineered sequences (e.g., two edited B2M alleles). Examples are shown in FIG. 5A, FIG. 5B, and FIG. 5C. For example, the cell can include a first genetically engineered sequence and a second genetically engineered sequence, wherein the first genetically engineered sequence is located on a first chromosome of the cell and the second genetically engineered sequence is located on a second chromosome of the cell. In a first example, the first genetically engineered sequence includes a first B2M:HLA fusion gene, a first multicistronic element, and a first kill switch gene; and the second genetically engineered sequence includes a second B2M:HLA fusion gene, a second multicistronic element, and a second kill switch gene. In a first specific example, the first B2M:HLA fusion gene is a B2M:HLA-E fusion gene, and the second B2M:HLA fusion gene is a B2M:HLA-G fusion gene. In a second specific example, the first and second B2M:HLA fusion genes are B2M:HLA-E fusion genes. In a third specific example, the first and second B2M:HLA fusion genes are B2M:HLA-G fusion genes. In a second example, the first genetically engineered sequence includes a B2M:HLA fusion gene (e.g., B2M:HLA-E fusion gene, B2M:HLA-G fusion gene, etc.), a multicistronic element, and a kill switch gene; and the second genetically engineered sequence includes a B2M knockout gene (e.g., the native B2M gene is edited such that the B2M gene is nonfunctional).

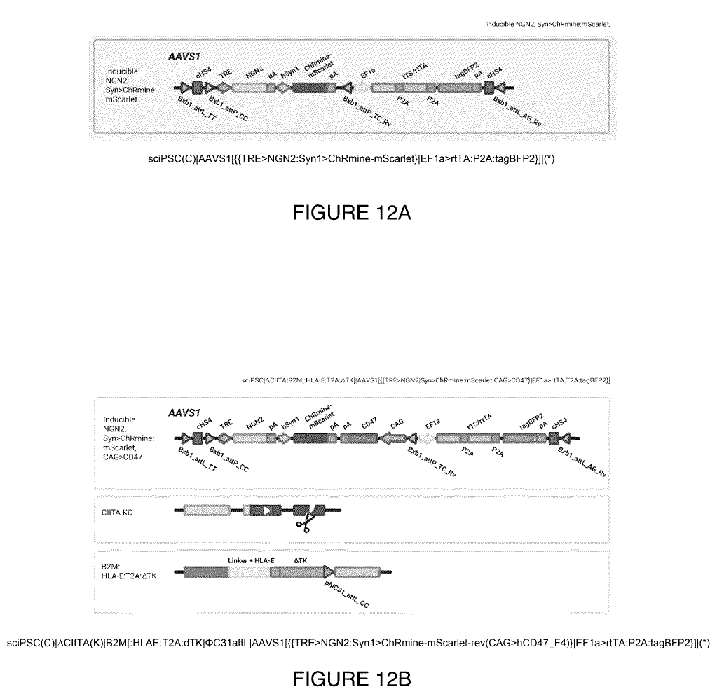

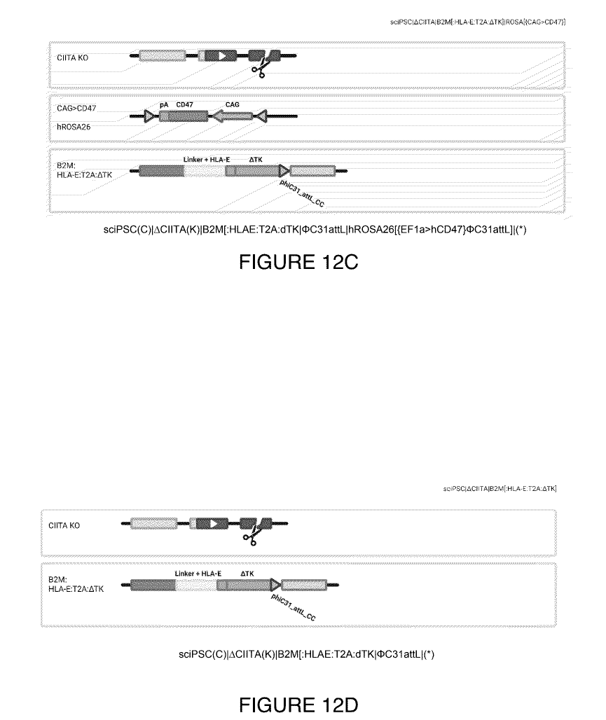

The genetically engineered sequence can optionally include one or more of the sequences set forth in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, and/or SEQ ID NO: 16. The genetically engineered sequence can optionally include any subsequence of one or more of the sequences set forth in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, and/or SEQ ID NO: 16. In some cases, the inducible promoter system includes a nucleotide sequence that differs by 1, 2, 3, 4, or 5 nucleotides from any of the sequences set forth in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, and/or SEQ ID NO: 16. In specific examples, the genetically engineered sequence can include sequences for the examples shown in FIG. 12A, FIG. 12B, FIG. 12C, or FIG. 12D. In a first specific example, a cell (e.g., the cell depicted in FIG. 12A) can include SEQ ID NO:1. In a second specific example, a cell (e.g., the cell depicted in FIG. 12A) can include SEQ ID NO:5. In a third specific example, a cell (e.g., the cell depicted in FIG. 12B) can include SEQ ID NO:2 and SEQ ID NO:4 (e.g., as noncontiguous sequences in the genome of the cell). In a fourth specific example, a cell (e.g., the cell depicted in FIG. 12B) can include SEQ ID NO:6 and SEQ ID NO:8. In a fifth specific example, a cell (e.g., the cell depicted in FIG. 12C) can include SEQ ID NO: 2 and SEQ ID NO:3 (e.g., as noncontiguous sequences in the genome of the cell). In a sixth specific example, a cell (e.g., the cell depicted in FIG. 12C) can include SEQ ID NO: 6 and SEQ ID NO:7. In a seventh specific example, a cell (e.g., the cell depicted in FIG. 12D) can include SEQ ID NO:2. In an eighth specific example, a cell (e.g., the cell depicted in FIG. 12D) can include SEQ ID NO:6.

The genetically engineered sequence can optionally include any or all of the sequences as described in U.S. application Ser. No. 18/662,102 filed 13 May 2024, titled “MODULAR GENETICALLY ENGINEERED CELL AND METHODS OF GENERATION THEREOF,” claiming priority to U.S. Provisional Application No. 63/465,759 filed 11 May 2023, each of which is incorporated in its entirety by this reference. In a specific example, the genetically engineered sequence can include one or more landing sites.

In an example, a cell (e.g., a genetically engineered cell) can be a CIITA knockout cell. In a specific example, the cell prior to knocking out the CIITA gene can include the sequence forth in SEQ ID NO: 9. In another specific example, the cell after knocking out the CIITA gene can include a sequence has at least 70%, 75%, 80%, 85%, 90%, 91%, 92%, 93%, 94%, 95%, 96%, 97%, 98%, or 99%, sequence identity with SEQ ID NO: 10.

In an example, a cell (e.g., a genetically engineered cell) can include a nucleotide sequence encoding CD47. The nucleotide sequence encoding CD47 can optionally be integrated into a safe harbor locus of the cell. In a specific example, the nucleotide sequence encoding CD47 can be integrated (e.g., via homology-directed repair) into a rosa26 locus of a genome of the cell. In a specific example, the cell can include the sequence forth in SEQ ID NO: 11 or SEQ ID NO: 12. In another specific example, the cell can include a sequence has at least 70%, 75%, 80%, 85%, 90%, 91%, 92%, 93%, 94%, 95%, 96%, 97%, 98%, or 99%, sequence identity with SEQ ID NO: 11 or SEQ ID NO: 12. The nucleotide sequence can optionally include a selection marker (e.g., tagBFP2) flanked by a pair of recombination sites corresponding to a site-specific recombinase (e.g., where the site-specific recombinase can be used to remove the selection marker). In a specific example, the cell before removal of the selection marker can include SEQ ID NO: 11 or a sequence has at least 70%, 75%, 80%, 85%, 90%, 91%, 92%, 93%, 94%, 95%, 96%, 97%, 98%, or 99%, sequence identity with SEQ ID NO: 11. In a specific example, the cell after removal of the selection marker (e.g., via PhiC31) can include SEQ ID NO: 12 or a sequence has at least 70%, 75%, 80%, 85%, 90%, 91%, 92%, 93%, 94%, 95%, 96%, 97%, 98%, or 99%, sequence identity with SEQ ID NO: 12.

In an example, a cell (e.g., a genetically engineered cell) can include a nucleotide sequence encoding a channelrhodopsin. The channelrhodopsin can optionally include an engineered WiChR variant (e.g., one or more mutations of wild-type WiChR). WiChR is a channelrhodopsin that occurs naturally in Wobblia lunata, a microscopic marine flagellate. The wild-type WiChR sequence is set forth in set forth in SEQ ID NO: 17. The engineered WiChR variant can have one or more advantages over the wild-type WiChR. In specific examples, the WiChR variant can have higher sensitivity (e.g., low light sensitivity), have reduced or abolished channelrhodopsin selectivity for potassium ions, greater expression efficiency, greater stability, greater functional selectivity, and/or any other enhanced performance characteristics. In a specific example, the engineered WiChR variant can include one or more mutations that functionally convert WiChR from an inhibitory optogenetic tool into an excitatory optogenetic tool. In one embodiment, a point mutation can be introduced at residue 240, changing Phenylalanine to Alanine. This mutation is designated as F240A, and the WiChR derivative having this mutation is designated as WiChR F240A. The F240A mutation preserves other functional properties of WiChR and is readily combined with other mutations. The engineered WiChR variant preferably includes the F240A mutation and optionally one or more additional mutations, but can alternatively include any other mutations. Indeed, the inventors have determined that the F240A mutation, combined with other mutations from the wild-type WiChR sequence, can enhance the functional properties of WiChR F240A and/or provide additional desirable properties to the resulting protein. For example, the inventors have determined that a modified WiChR, designated as WiChR S122L/F240A (Serine to Leucine at residue 122, Phenylalanine to Alanine at residue 240) can have an increased light sensitivity. In a specific example, the engineered WiChR variant can include one or more mutations of the wild-type WiChR sequence while maintaining at least 70%, 75%, 80%, 85%, 90%, 91%, 92%, 93%, 94%, 95%, 96%, 97%, 98%, or 99% sequence identity to the wild-type WiChR sequence. For example, the engineered WiChR variant can have at least 70%, 75%, 80%, 85%, 90%, 91%, 92%, 93%, 94%, 95%, 96%, 97%, 98%, or 99%, sequence identity with SEQ ID NO: 17.

The nucleotide sequence encoding the channelrhodopsin can optionally be integrated into a safe harbor locus of the cell. In a specific example, the nucleotide sequence encoding the channelrhodopsin can be integrated (e.g., via homology-directed repair) into an AAVS1 locus of a genome of the cell. The nucleotide sequence encoding the channelrhodopsin can optionally additionally include an MMP2 gene. All or a portion of the nucleotide sequence encoding the channelrhodopsin can optionally be doxycycline inducible (e.g., the sequence can include a doxycycline inducible promoter). In a specific example, the cell can include the sequence forth in SEQ ID NO: 13 or SEQ ID NO: 14. In another specific example, the cell can include a sequence has at least 70%, 75%, 80%, 85%, 90%, 91%, 92%, 93%, 94%, 95%, 96%, 97%, 98%, or 99%, sequence identity with SEQ ID NO: 13 or SEQ ID NO: 14. The nucleotide sequence can optionally include a selection marker (e.g., eGFP) flanked by a pair of recombination sites corresponding to a site-specific recombinase (e.g., where the site-specific recombinase can be used to remove the selection marker). In a specific example, the cell before removal of the selection marker can include SEQ ID NO: 13 or a sequence has at least 70%, 75%, 80%, 85%, 90%, 91%, 92%, 93%, 94%, 95%, 96%, 97%, 98%, or 99%, sequence identity with SEQ ID NO: 13. In a specific example, the cell after removal of the selection marker (e.g., via PhiC31) can include SEQ ID NO: 14 or a sequence has at least 70%, 75%, 80%, 85%, 90%, 91%, 92%, 93%, 94%, 95%, 96%, 97%, 98%, or 99%, sequence identity with SEQ ID NO: 14.

In an example, a cell (e.g., a genetically engineered cell) can include a nucleotide sequence that includes: a B2M:HLA fusion gene, a multicistronic element and a kill switch gene, wherein the multicistronic element is located between the kill switch gene and the B2M:HLA fusion gene. This edit can optionally be a homozygous edit. For example, the cell can include (on another chromosome of the cell) a second nucleotide sequence that includes a second B2M:HLA fusion gene, a second multicistronic element and a second kill switch gene, wherein the second multicistronic element is located between the second kill switch gene and the second B2M:HLA fusion gene. In a specific example, the cell can include the sequence forth in SEQ ID NO: 15 or SEQ ID NO: 16. In another specific example, the cell can include a sequence has at least 70%, 75%, 80%, 85%, 90%, 91%, 92%, 93%, 94%, 95%, 96%, 97%, 98%, or 99%, sequence identity with SEQ ID NO: 15 or SEQ ID NO: 16. The nucleotide sequence can optionally include a selection marker (e.g., eGFP) flanked by a pair of recombination sites corresponding to a site-specific recombinase (e.g., where the site-specific recombinase can be used to remove the selection marker). In a specific example, the cell before removal of the selection marker can include SEQ ID NO: 15 or a sequence has at least 70%, 75%, 80%, 85%, 90%, 91%, 92%, 93%, 94%, 95%, 96%, 97%, 98%, or 99%, sequence identity with SEQ ID NO: 15. In a specific example, the cell after removal of the selection marker (e.g., via PhiC31) can include SEQ ID NO: 16 or a sequence has at least 70%, 75%, 80%, 85%, 90%, 91%, 92%, 93%, 94%, 95%, 96%, 97%, 98%, or 99%, sequence identity with SEQ ID NO: 16. In a specific example, the B2M:HLA fusion gene is nonfunctional without removal of the selection marker.

The cell can optionally include multiple genetically engineered sequences (e.g., two or more of the nucleotide sequences as described above). For example, the cell can include two or more of: a first nucleotide sequence including a B2M:HLA fusion gene, a multicistronic element and a kill switch gene (e.g., wherein the multicistronic element is located between the kill switch gene and the B2M:HLA fusion gene); a second nucleotide sequence encoding a channelrhodopsin; and/or a third nucleotide sequence encoding CD47. Selection markers in one or more of the sequences can optionally be removed using the same type of site-specific recombinase (e.g., PhiC31). In a specific example, the combination of these two or more nucleotide sequences can enable the cell to be used for optogenetic applications within the human body (e.g., where the cell is implanted in the body). In a specific example, the cell can be used in an implant (e.g., a neural interface device, as described above).

However, a genetically engineered sequence can be otherwise configured, and/or a genetically engineered cell can be otherwise created.

### 6. Specific Examples

A numbered list of specific examples of the technology described herein are provided below. A person of skill in the art will recognize that the scope of the technology is not limited to and/or by these specific examples.

Specific Example 1: A genetically engineered cell, comprising: a genetically engineered sequence comprising: a Beta-2-Microglobulin (B2M): human leukocyte antigen (HLA) fusion gene; a multicistronic element contiguous with the B2M:HLA fusion gene; and a kill switch gene contiguous with the multicistronic element.

Specific Example 2: The genetically engineered cell of Specific Example 1, wherein the kill switch gene and the B2M-HLA fusion gene are each located in a shared open reading frame, wherein the genetically engineered sequence generates a B2M:HLA fusion protein and a kill switch protein, the kill switch protein separate from the B2M:HLA fusion protein.

Specific Example 3: The genetically engineered cell of any of Specific Examples 1 or 2, wherein the multicistronic element is located between the kill switch gene and the B2M:HLA fusion gene.

Specific Example 4: The genetically engineered cell of any of Specific Examples 1-3, wherein the genetically engineered sequence further comprises a non-native B2M sequence replacing a B2M sequence native to the genetically engineered cell.

Specific Example 5: The genetically engineered cell of any of Specific Examples 1-4, further comprising a second genetically engineered sequence comprising: a second B2M:HLA fusion gene; a second multicistronic element; and a second kill switch gene; wherein the genetically engineered sequence is located on a first chromosome of the cell and the second genetically engineered sequence is located on a second chromosome of the cell.

Specific Example 6: The genetically engineered cell of Specific Example 5, wherein the first B2M:HLA fusion gene comprises a B2M:HLA-E fusion gene, and wherein the second B2M:HLA fusion gene comprises a B2M:HLA-G fusion gene

Specific Example 7: The genetically engineered cell of any of Specific Examples 1-6, wherein the multicistronic element and kill switch gene are located within a B2M exon 3 sequence of the B2M-HLA fusion gene.

Specific Example 8: The genetically engineered cell of any of Specific Examples 1-7, wherein the genetically engineered sequence is generated by simultaneously integrating an HLA gene, the multicistronic element, and the kill switch gene into a genome of the genetically engineered cell.

Specific Example 9: The genetically engineered cell of any of Specific Examples 1-8, wherein the kill switch gene comprises at least one of Delta-Thymidine Kinase, iCasp9, or RapaCasp.

Specific Example 10: The genetically engineered cell of any of Specific Examples 1-9, wherein the multicistronic element comprises at least one of: T2A, P2A, E2A, or F2A.

Specific Example 11: The genetically engineered cell of any of Specific Examples 1-10, wherein the genetically engineered cell comprises a genetically engineered human induced pluripotent stem cell.

Specific Example 12: A method, comprising: integrating an insertion sequence into a Beta-2-Microglobulin (B2M) gene of a cell, the insertion sequence comprising: a human leukocyte antigen (HLA) class I gene; a multicistronic element; and a kill switch gene, wherein the multicistronic element is located between the kill switch gene and the HLA class I gene in the insertion sequence.

Specific Example 13: The method of Specific Example 12, wherein the multicistronic element is contiguous with the HLA class I gene and is contiguous with kill switch gene.

Specific Example 14: The method of any of Specific Examples 12-13, wherein the insertion sequence further comprises a selection sequence comprising a pair of recombination sites bounding a selectable marker, the method further comprising excising the selectable marker using a recombinase.

Specific Example 15: The method of any of Specific Examples 12-14, wherein the insertion sequence further comprises a replacement B2M sequence, wherein integrating the insertion sequence into the B2M gene comprises replacing a native B2M sequence in the B2M gene with the replacement B2M sequence, wherein the replacement B2M sequence and the native B2M sequence are substantially identical.

Specific Example 16: The method of any of Specific Examples 12-15, wherein the insertion sequence is integrated within exon 3 of the B2M gene.

Specific Example 17: The method of any of Specific Examples 12-16, wherein the insertion sequence is located on a 5′ side of a stop codon of exon 3 of the B2M gene.

Specific Example 18: The method of any of Specific Examples 12-17, wherein the kill switch gene is co-transcribed with the HLA class I gene and the multicistronic element.

Specific Example 19: The method of any of Specific Examples 12-18, further comprising integrating a second insertion sequence within a second B2M gene of the cell, the second B2M gene located on a different chromosome than the B2M gene, the second insertion sequence comprising: a second HLA class I gene; a second multicistronic element; and a second kill switch gene.

Specific Example 20: The method of any of Specific Examples 12-19, wherein the HLA class I gene comprises at least one of an HLA-E gene or an HLA-G gene.

Specific Example 21: The method of any of Specific Examples 12-20, wherein the insertion sequence further comprises a linker between the replacement B2M sequence and the HLA class I gene.

Specific Example 22: The genetically engineered cell of any of Specific Examples 1-11, wherein the genetically engineered cell comprises a CIITA knockout cell.

Specific Example 23: A method for producing (e.g., generating, genetically engineering, etc.) the genetically engineered cell of any of Specific Examples 1-11 or 22.

Specific Example 24: A genetically engineered cell produced (e.g., generated, genetically engineered, etc.) by any of Specific Examples 12-21.

Specific Example 25: A genetically engineered cell, comprising: a first nucleotide sequence comprising: a Beta-2-Microglobulin (B2M): human leukocyte antigen (HLA) fusion gene, a multicistronic element and a kill switch gene, wherein the multicistronic element is located between the kill switch gene and the B2M:HLA fusion gene; and a second nucleotide sequence encoding a channelrhodopsin.

Specific Example 26: The genetically engineered cell of Specific Example 25, wherein the channelrhodopsin comprises an engineered WiChR variant.

Specific Example 27: The genetically engineered cell of any of Specific Examples 25-26, wherein the second nucleotide sequence further comprises an MMP2 gene.

Specific Example 28: The genetically engineered cell of any of Specific Examples 25-27, wherein the second nucleotide sequence further comprises a doxycycline inducible promoter.

Specific Example 29: The genetically engineered cell of any of Specific Examples 25-28, wherein the second nucleotide sequence is integrated into a AAvs1 locus of a genome of the genetically engineered cell.

Specific Example 30: The genetically engineered cell of any of Specific Examples 25-29, further comprising a third nucleotide sequence comprising: a second B2M:HLA fusion gene, a second multicistronic element and a second kill switch gene, wherein the second multicistronic element is located between the second kill switch gene and the second B2M:HLA fusion gene, the first nucleotide sequence located on a first chromosome of the genetically engineered cell, and the third nucleotide sequence located on a second chromosome of the genetically engineered cell.

Specific Example 31: The genetically engineered cell of any of Specific Examples 25-30, wherein the first nucleotide sequence has at least 70% sequence identity with SEQ ID NO: 15 or SEQ ID NO: 16.

Specific Example 32: The genetically engineered cell of any of Specific Examples 25-31, wherein the second nucleotide sequence has at least 70% sequence identity with SEQ ID NO: 13 or SEQ ID NO: 14.

Specific Example 33: The genetically engineered cell of any of Specific Examples 25-32, wherein the first nucleotide sequence comprises the nucleotide sequence of SEQ ID NO: 15 or SEQ ID NO: 16.

Specific Example 34: The genetically engineered cell of any of Specific Examples 25-33, wherein the second nucleotide sequence comprises the nucleotide sequence of SEQ ID NO: 13 or SEQ ID NO: 14.

Specific Example 35: The genetically engineered cell of any of Specific Examples 25-34, further comprising a third nucleotide sequence comprising a CD47 gene.

Specific Example 36: The genetically engineered cell of Specific Example 35, wherein the third nucleotide sequence is integrated into a rosa26 locus of a genome of the genetically engineered cell.

Specific Example 37: The genetically engineered cell of any of Specific Examples 35-36, wherein the first nucleotide sequence comprises a first selection marker flanked by a first pair of pair of recombination sites corresponding to a site-specific recombinase, wherein the second nucleotide sequence comprises a second selection marker flanked by a second pair of recombination sites corresponding to the site-specific recombinase, and wherein the third nucleotide sequence comprises a third selection marker flanked by a third pair of recombination sites corresponding to the site-specific recombinase.

Specific Example 38: The genetically engineered cell of any of Specific Examples 35-37, wherein the third nucleotide sequence comprises the nucleotide sequence of SEQ ID NO: 11 or SEQ ID NO: 12.

Specific Example 39: The genetically engineered cell of any of Specific Examples 25-38, wherein a CIITA gene of the genetically engineered cell is knocked out.

Specific Example 40: The genetically engineered cell of any of Specific Examples 25-39, wherein the genetically engineered cell is a human induced pluripotent stem cell.

Specific Example 41: The genetically engineered cell of any of Specific Examples 25-39, wherein the genetically engineered cell is a neuron.

Specific Example 42: A neural interface device configured to be implanted on a surface of a brain, the neural interface device comprising a set of scaffolds and the genetically engineered cell of any of Specific Examples 25-41, wherein the genetically engineered cell is seeded between two scaffolds of the set of scaffolds.

Specific Example 43: The neural interface device of Specific Example 42, wherein the set of scaffolds comprises: a recording electrode configured to receive an electrical signal from the genetically engineered cell; and a μLED configured to transmit a light signal to the genetically engineered cell, wherein the light signal activates the channelrhodopsin.

Specific Example 44: The neural interface device of any of Specific Examples 42-43, wherein the first nucleotide sequence has at least 70% sequence identity with SEQ ID NO: 15 or SEQ ID NO: 16, and wherein the second nucleotide sequence has at least 70% sequence identity with SEQ ID NO: 13 or SEQ ID NO: 14.

It is contemplated that the sequences disclosed herein may tolerate minor variations while retaining their functional properties. Such variations may include conservative substitutions, insertions, deletions, or modifications that do not materially alter the structure, stability, or activity of the encoded nucleic acid or protein. Accordingly, sequences having at least 70%, 75%, 80%, 85%, 90%, 91%, 92%, 93%, 94%, 95%, 96%, 97%, 98%, or 99% identity to any of the disclosed sequences are considered within the scope of the invention. Sequence percent identity may be determined using standard alignment algorithms such as BLAST, ClustalW, or other suitable computational methods known in the art. Variants that retain substantially equivalent biological activity, function, or expression characteristics to the disclosed sequences are encompassed by the present invention, including sequences that exhibit equivalent functional properties, binding properties, folding properties, and/or configurations.

It is further contemplated that specific point mutations disclosed herein in accordance with a sequence according to one embodiment may be combined in various permutations to generate additional sequence variants that retain the functional properties of the disclosed sequences. Accordingly, one or more point mutations described in a given sequence may be combined with one or more point mutations from another disclosed sequence to produce a variant that remains within the scope of the invention. Such combinations may result in sequences having modified but functionally equivalent characteristics, including but not limited to altered stability, expression levels, or functional activity as described herein. Variants generated by combining disclosed mutations while maintaining at least 70%, 75%, 80%, 85%, 90%, 91%, 92%, 93%, 94%, 95%, 96%, 97%, 98%, or 99% sequence identity to an original disclosed sequence are expressly encompassed within the invention.

As used herein, “substantially” or other words of approximation (e.g., “about,” “approximately,” etc.) can be within a predetermined error threshold or tolerance of a metric, component, or other reference (e.g., within +/−0.001%, +/−0.01%, +/−0.1%, +/−1%, +/−2%, +/−5%, +/−10%, +/−15%, +/−20%, +/−30%, any range or value therein, of a reference).

All references cited herein are incorporated by reference in their entirety, except to the extent that the incorporated material is inconsistent with the express disclosure herein, in which case the language in this disclosure controls.

Embodiments of the system and/or method can include every combination and permutation of the various system components and the various method processes, wherein one or more instances of the method and/or processes described herein can be performed asynchronously (e.g., sequentially), contemporaneously (e.g., concurrently, in parallel, etc.), or in any other suitable order by and/or using one or more instances of the systems, elements, and/or entities described herein. Components and/or processes of the following system and/or method can be used with, in addition to, in lieu of, or otherwise integrated with all or a portion of the systems and/or methods disclosed in the applications mentioned above, each of which are incorporated in their entirety by this reference.

As a person skilled in the art will recognize from the previous detailed description and from the figures and claims, modifications and changes can be made to the preferred embodiments of the invention without departing from the scope of this invention defined in the following claims.

We claim:

1. A genetically engineered cell, comprising:
a first nucleotide sequence comprising a Beta-2-Microglobulin (B2M):human leukocyte antigen (HLA) fusion gene, a multicistronic element and a kill switch gene, wherein the multicistronic element is located between the kill switch gene and the B2M:HLA fusion gene; and
a second nucleotide sequence encoding a channelrhodopsin.

2. The genetically engineered cell of claim 1, wherein the channelrhodopsin comprises an engineered WiChR variant.

3. The genetically engineered cell of claim 2, wherein the second nucleotide sequence further comprises an MMP2 gene.

4. The genetically engineered cell of claim 3, wherein the second nucleotide sequence further comprises a doxycycline inducible promoter.

5. The genetically engineered cell of claim 2, wherein the second nucleotide sequence is integrated into a AAvs1 locus of a genome of the genetically engineered cell.

6. The genetically engineered cell of claim 1, further comprising a third nucleotide sequence comprising: a second B2M:HLA fusion gene, a second multicistronic element and a second kill switch gene, wherein the second multicistronic element is located between the second kill switch gene and the second B2M:HLA fusion gene, the first nucleotide sequence located on a first chromosome of the genetically engineered cell, and the third nucleotide sequence located on a second chromosome of the genetically engineered cell.

7. The genetically engineered cell of claim 1, wherein the first nucleotide sequence has at least 70% sequence identity with SEQ ID NO: 15 or SEQ ID NO: 16.

8. The genetically engineered cell of claim 7, wherein the second nucleotide sequence has at least 70% sequence identity with SEQ ID NO: 13 or SEQ ID NO: 14.

9. The genetically engineered cell of claim 1, wherein the first nucleotide sequence comprises the nucleotide sequence of SEQ ID NO: 15 or SEQ ID NO: 16.

10. The genetically engineered cell of claim 9, wherein the second nucleotide sequence comprises the nucleotide sequence of SEQ ID NO: 13 or SEQ ID NO: 14.

11. The genetically engineered cell of claim 1, further comprising a third nucleotide sequence comprising a CD47 gene.

12. The genetically engineered cell of claim 11, wherein the third nucleotide sequence is integrated into a rosa26 locus of a genome of the genetically engineered cell.

13. The genetically engineered cell of claim 11, wherein the first nucleotide sequence comprises a first selection marker flanked by a first pair of pair of recombination sites corresponding to a site-specific recombinase, wherein the second nucleotide sequence comprises a second selection marker flanked by a second pair of recombination sites corresponding to the site-specific recombinase, and wherein the third nucleotide sequence comprises a third selection marker flanked by a third pair of recombination sites corresponding to the site-specific recombinase.

14. The genetically engineered cell of claim 11, wherein the third nucleotide sequence comprises the nucleotide sequence of SEQ ID NO: 11 or SEQ ID NO: 12.

15. The genetically engineered cell of claim 1, wherein a CIITA gene of the genetically engineered cell is knocked out.

16. The genetically engineered cell of claim 1, wherein the genetically engineered cell is a human induced pluripotent stem cell.

17. The genetically engineered cell of claim 1, wherein the genetically engineered cell is a neuron.

18. A neural interface device configured to be implanted on a surface of a brain, the neural interface device comprising a set of scaffolds and a genetically engineered cell, wherein the genetically engineered cell is seeded between two scaffolds of the set of scaffolds, the genetically engineered cell comprising:
a first nucleotide sequence comprising: a Beta-2-Microglobulin (B2M):human leukocyte antigen (HLA) fusion gene, a multicistronic element and a kill switch gene, wherein the multicistronic element is located between the kill switch gene and the B2M:HLA fusion gene; and
a second nucleotide sequence encoding a channelrhodopsin.

19. The neural interface device of claim 18, wherein the set of scaffolds comprises:
a recording electrode configured to receive an electrical signal from the genetically engineered cell; and
a μLED configured to transmit a light signal to the genetically engineered cell, wherein the light signal activates the channelrhodopsin.

20. The neural interface device of claim 18, wherein the first nucleotide sequence has at least 70% sequence identity with SEQ ID NO: 15 or SEQ ID NO: 16, and wherein the second nucleotide sequence has at least 70% sequence identity with SEQ ID NO: 13 or SEQ ID NO: 14.
